SEQUENCE LISTING (8 sequences)

SEQ ID NO: 1 - protein, 116 aa, Artificial Sequence
  Description of Artificial Sequence Synthetic polypeptide
RVTKDAETEFMMSKLPLENPVLLDMLWRRKIGPQMTLSHAAGFHATSADCCISYTPRSIPCSLLESYFETNSECSKPGVIFLTKKGRRFCANPSDKQVQVCMRMLKLDTRIKTRKN

SEQ ID NO: 2 - protein, 99 aa, Artificial Sequence
  Description of Artificial Sequence Synthetic polypeptide
RVTKDAETEFMMSKLPLENPVLLDRFHATSADCCISYTPRSIPCSLLESYFETNSECSKPGVIFLTKKGRRFCANPSDKQVQVCMRMLKLDTRIKTRKN

SEQ ID NO: 3 - protein, 18 aa, Artificial Sequence
  Description of Artificial Sequence Synthetic peptide
MLWRRKIGPQMTLSHAAG

SEQ ID NO: 4 - protein, 21 aa, Artificial Sequence
  Description of Artificial Sequence Synthetic peptide
MKVSVAALSCLMLVTALGSQA

SEQ ID NO: 5 - protein, 108 aa, Artificial Sequence
  Description of Artificial Sequence Synthetic polypeptide
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 6 - protein, 134 aa, Artificial Sequence
  Description of Artificial Sequence Synthetic polypeptide
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 7 - protein, 19 aa, Artificial Sequence
  Description of Artificial Sequence Synthetic peptide
MLWRRKIGPQMTLSHAAGC

SEQ ID NO: 8 - protein, 9 aa, Artificial Sequence
  Description of Artificial Sequence Synthetic peptide
RFHATSADC

(19) United States
(12) Patent Application Publication
(10) Pub. No.: US 2009/0004755 A1
(43) Pub. Date: Jan. 1, 2009

(54) METHODS AND COMPOSITIONS FOR DIAGNOSIS AND/OR PROGNOSIS IN SYSTEMIC INFLAMMATORY RESPONSE SYNDROMES

(75) Inventors: Seok-Won Lee, San Diego, CA (US); Kelline M. Rodems, Oceanside, CA (US); David W. Oelschlager, San Diego, CA (US); Uday Kumar Veeramallu, San Diego, CA (US); Joseph A. Buechler, Carlsbad, CA (US); Paul H. McPherson, Encinitas, CA (US)

Correspondence Address: Inverness Medical Innovations / WSGR;Wilson Sonsini Goodrich & Rosati, P.C., 650 Page Mill Road, Palo Alto, CA, 94304, US

(73) Assignee: Biosite, Incorporated

(21) Appl. No.: 11/770,608

(22) Filed: Jun. 28, 2007

Related U.S. Application Data
(63) Continuation-in-part of application No. 11/690,767, filed on Mar. 23, 2007.

(51) Int. Cl. G01N 33/566

(52) U.S. Cl. 436/501

(57) ABSTRACT

The present invention relates to methods and compositions for diagnosing SIRS, sepsis, severe sepsis, septic shock, or MODS in a subject, or assigning a prognostic risk for one or more clinical outcomes for a subject suffering from SIRS, sepsis, severe sepsis, septic shock, or MODS, the method comprising performing an immunoassay for CCL23 splice variant.

### CROSS-REFERENCE TO RELATED PATENT APPLICATIONS

This application is a continuation in part of U.S. application Ser. No. 11/690,767, filed Mar. 23, 2007, which is incorporated by reference herein its entirety, including all tables, figures, and claims. This application is related to U.S. application Ser. No. 11/543,312, filed Oct. 3, 2006, and U.S. application Ser. No. 11/022,552, filed Dec. 23, 2004, each of which is incorporated by reference herein its entirety, including all tables, figures, and claims.

### FIELD OF THE INVENTION

The present invention relates to the identification and use of diagnostic markers related to sepsis. In a various aspects, the invention relates to methods and compositions for use in assigning a treatment pathway to subjects suffering from SIRS, sepsis, severe sepsis, septic shock and/or multiple organ dysfunction syndrome.

### BACKGROUND OF THE INVENTION

The following discussion of the background of the invention is merely provided to aid the reader in understanding the invention and is not admitted to describe or constitute prior art to the present invention.

The term “sepsis” has been used to describe a variety of clinical conditions related to systemic manifestations of inflammation accompanied by an infection. Because of clinical similarities to inflammatory responses secondary to non-infectious etiologies, identifying sepsis has been a particularly challenging diagnostic problem. Recently, the American College of Chest Physicians and the American Society of Critical Care Medicine (Bone et al., Chest 101: 1644-53, 1992) published definitions for “Systemic Inflammatory Response Syndrome” (or “SIRS”), which refers generally to a severe systemic response to an infectious or non-infectious insult, and for the related syndromes “sepsis,” “severe sepsis,” and “septic shock,” and extending to multiple organ dysfunction syndrome (“MODS”). These definitions, described below, are intended for each of these phrases for the purposes of the present application. For purposes of this invention, each of these represents a progressively more severe SIRS category; that is, sepsis is more severe than SIRS, severe sepsis is more severe than sepsis, septic shock is more severe than severe sepsis, and MODS is more severe than septic shock.

“SIRS” refers to a condition that exhibits two or more of the following:

a temperature>38° C. or <36° C.;
a heart rate of >90 beats per minute (tachycardia);
a respiratory rate of >20 breaths per minute (tachypnea) or a PₐCO₂<4.3 kPa; and
a white blood cell count>12,000 per mm³, <4,000 per mm³, or >10% immature (band) forms.

“Sepsis” refers to SIRS, further accompanied by a clinically evident or microbiologically confirmed infection. This infection may be bacterial, fungal, parasitic, or viral.

“Severe sepsis” refers to sepsis, further accompanied by organ hypoperfusion made evident by at least one sign of organ dysfunction such as hypoxemia, oliguria, metabolic acidosis, or altered cerebral function.

“Septic shock” refers to severe sepsis, further accompanied by hypotension, made evident by a systolic blood pressure<90 mm Hg, or the requirement for pharmaceutical intervention to maintain blood pressure.

MODS (multiple organ dysfunction syndrome) is the presence of altered organ function in a patient who is acutely ill such that homeostasis cannot be maintained without intervention. Primary MODS is the direct result of a well-defined insult in which organ dysfunction occurs early and can be directly attributable to the insult itself. Secondary MODS develops as a consequence of a host response and is identified within the context of SIRS.

A systemic inflammatory response leading to a diagnosis of SIRS may be related to both infection and to numerous non-infective etiologies, including burns, pancreatitis, trauma, heat stroke, and neoplasia. While conceptually it may be relatively simple to distinguish between sepsis and non-septic SIRS, no diagnostic tools have been described to unambiguously distinguish these related conditions. See, e.g., Llewelyn and Cohen, Int. Care Med. 27: S10-S32, 2001. For example, because more than 90% of sepsis cases involve bacterial infection, the “gold standard” for confirming infection has been microbial growth from blood, urine, pleural fluid, cerebrospinal fluid, peritoneal fluid, synnovial fluid, sputum, or other tissue specimens. Such culture has been reported, however, to fail to confirm 50% or more of patients exhibiting strong clinical evidence of sepsis. See, e.g., Jaimes et al., Int. Care Med 29: 1368-71, published electronically Jun. 26, 2003.

The physiologic responses leading to the systemic manifestations of inflammation in sepsis remain unclear. Activation of immune cells occurs in response to the LPS endotoxin of gram negative bacteria and exotoxins of gram positive bacteria. This activation leads to a cascade of events mediated by proinflammatory cytokines, adhesion molecules, vasoactive mediators, and reactive oxygen species. Various organs, including the liver, lungs, heart, and kidney are affected directly or indirectly by this cascade. Sepsis is also associated with disseminated intravascular coagulation (“DIC”), mediated presumably by cytokine activation of coagulation. Fluid and electrolyte balance are also affected by increases in capillary perfusion and reduced oxygenation of tissues. Unchecked, the uncontrolled inflammatory response created can lead to ischemia, loss of organ function, and death.

Despite the availability of antibiotics and supportive therapy, sepsis represents a significant cause of morbidity and mortality. A recent study estimated that 751,000 cases of severe sepsis occur in the United States annually, with a mortality rate of from 30-50%. Angus et al., Crit. Care Med. 29: 1303-10, 2001. Recently, an organization of medical care groups referred to as the “Surviving Sepsis Campaign” issued guidelines for managing subjects suffering from severe sepsis and septic shock. Dellinger et al., Crit. Care Med. 32: 858-873, 2004. These guidelines draw from, amongst other sources, the “Early Goal Directed Therapy” therapy regimen developed by Rivers and colleagues. See, e.g., New Engl. J. Med. 345: 1368-77. 2001.

Several laboratory tests have been investigated or proposed for use, in conjunction with a complete clinical examination of a subject, for the diagnosis and prognosis of sepsis. See, e.g., U.S. Pat. Nos. 5,639,617 and 6,303,321; Patent publications US2005/0196817, WO2005/048823, WO2004/046181, WO2004/043236, US2005/0164238; and Charpentier et al., Crit. Care Med. 32: 660-65, 2004; Castillo et al., Int. J. Infect. Dis. 8: 271-74, 2004; Chua and Kang-Hoe, Crit. Care 8: R248-R250, 2004; Witthaut et al., Int. Care Med. 29: 1696-1702, 2003; Jones and Kline, Ann. Int. Med. 42: 714-15, 2003; Maeder et al., Swiss Med. Wkly. 133: 515-18, 2003; Giamarellos-Bourboulis et al., Intensive Care Med. 28: 1351-56, 2002; Harbarth et al., Am. J. Respir. Crit. Care Med. 164: 396-402, 2001; Martin et al., Pediatrics 108: (4) e61 1-6, 2001; and Bossink et al., Chest 113: 1533-41, 1998. The use of CCL23 as a marker in sepsis is disclosed in US 2005/0196817 (where it is called by its alternative name MPIF-1) and in WO07/041,623.

### BRIEF SUMMARY OF THE INVENTION

The present invention relates to the identification and use of markers for the detection of sepsis, the differentiation of sepsis from other causes of SIRS, and in the stratification of risk in sepsis patients. The methods and compositions of the present invention can be used to facilitate the treatment of patients and the development of additional diagnostic and/or prognostic indicators and therapies.

In various aspects, the invention relates to materials and procedures for identifying markers that may be used to direct therapy in subjects; to using such markers in treating a patient and/or to monitor the course of a treatment regimen; to using such markers to identify subjects at risk for one or more adverse outcomes related to SIRS; and for screening compounds and pharmaceutical compositions that might provide a benefit in treating or preventing such conditions.

In a first aspect, the invention relates to diagnostic methods for identifying a subject suffering from SIRS, sepsis, severe sepsis, septic shock and/or MODS, for distinguishing amongst these conditions, or for assigning a prognosis to a subject suffering from one or more of these conditions. These methods comprise analyzing a test sample obtained from a subject by performing an immunoassay that detects CCL23 splice variant; and relating the immunoassay result to one or more of the following diagnoses: (i) the presence or absence of SIRS, (ii) the presence or absence of sepsis, (iii) the presence or absence of severe sepsis, and (iv) the presence or absence of septic shock. The terms “CCL23 splice variant” and “CCL23” are defined hereinafter.

In a related aspect, the invention relates to methods for distinguishing among SIRS, sepsis, severe sepsis, septic shock and/or MODS. These methods similarly comprise analyzing a test sample obtained from a subject by performing an immunoassay that detects CCL23 splice variant; and relating the immunoassay result to ruling in or out one or more of the following diagnoses: that the subject has SIRS, but not sepsis, severe sepsis, or septic shock; that the subject has sepsis, but not severe sepsis or septic shock; or that the subject has septic shock.

In a related aspect, the invention relates to methods for determining a prognosis for a subject suffering from SIRS, sepsis, severe sepsis, septic shock and/or MODS. These methods similarly comprise analyzing a test sample obtained from a subject by performing an immunoassay that detects CCL23 splice variant; and relating the immunoassay result to the likelihood of a future outcome, either positive (e.g., that the subject is more likely to live, or is at a decreased risk of progressing to a more severe SIRS category) or negative (e.g., that the subject is at an increased risk of death, that the subject is at an increased risk of progressing to a more severe SIRS category).

And in still another related aspect, the invention relates to a method of monitoring a treatment regimen in a subject being treated for SIRS, sepsis, severe sepsis, septic shock and/or MODS. These methods similarly comprise analyzing a test sample obtained from a subject by performing an immunoassay that detects CCL23 splice variant; and relating the immunoassay result to the success or failure of the treatment received by the subject.

As described herein, preferred assays are “configured to detect” CCL23 splice variant, which means that the assay can generate a detectable signal indicative of the presence or amount of a physiologically relevant concentration of CCL23 splice variant. As described hereinafter, such assays may also detect CCL23.

Assays may be configured to not appreciably detect CCL23, thereby providing an immunoassay result that is sensitive for CCL23 splice variant, relative to CCL23 itself. These are referred to herein as “CCL23 splice variant immunoassays” or “CCL23sv immunoassays.” In preferred embodiments, however, the immunoassay that detects CCL23 splice variant also detects CCL23, and optionally detects one or more of, and optionally each of, N-terminal processed forms of CCL23 selected from the group consisting of CCL23₁₉₋₉₉, CCL23₂₂₋₉₉, CCL23₂₇₋₉₉, and CCL23₃₀₋₉₉. These assays, which detect both CCL23 splice variant and CCL23, are referred to herein as “total CCL23 immunoassays” and the results obtained therefrom are refereed to as “total CCL23 assay results.” Total CCL23 immunoassays that do not recognize one or more N-terminal processed forms of CCL23 selected from the group consisting of CCL23₁₉₋₉₉, CCL23₂₂₋₉₉, CCL23₂₇₋₉₉, and CCL23₃₀₋₉₉. are referred to herein as “full length CCL23 immunoassays.” Each of these labels is used here for convenience in referring to the various assays, and is not meant to be fully descriptive of such assays. For example, the phrase “full length CCL23 immunoassay” is not meant to imply that such assays necessarily recognize only CCL23₁₋₉₉.

Collectively, the assays configured to detect CCL23 splice variant, whether a CCL23sv immunoassay or a total CCL23 immunoassay, are referred to herein as “CCL23 assays” and the results obtained therefrom are referred to as CCL23 assay results.” For the sake of clarity, assays that detect CCL23 but do not appreciably detect CCL23 splice variant are not “CCL23 assays” as that term is used herein. If referred to, such assays will be referred to as being “CCL23-specific.”

The CCL23 immunoassays may be described as being “sensitive” or “insensitive” for CCL23 splice variant, relative to CCL23. “Sensitive” assays, as that term is used herein, are configured to provide a signal that is at least a factor of 5, more preferably a factor of ten, and most preferably a factor of 100 or more, greater for CCL23 splice variant at its physiologically relevant concentration as compared to equimolar amounts of CCL23. In the case of assays that are sensitive for CCL23 splice variant, relative to CCL23, such assays preferably employ one or more antibodies that specifically bind CCL23 splice variant, relative to CCL23. As such, the affinity of one or more antibodies used in the immunoassay is at least 5 fold, preferably 10 fold, more preferably 25-fold, even more preferably 50-fold, and most preferably 100-fold or more, greater for CCL23 splice variant than its affinity for CCL23. Such antibodies are preferably directed to an epitope that is present on CCL23 splice variant, but not on CCL23 itself.

“Insensitive” assays, as that term is used herein, are configured to provide a signal that is within a factor of 2, and most preferably a factor of 0.5 or less, for CCL23 splice variant at its physiologically relevant concentration as compared to equimolar amounts of CCL23. Such assays may preferably be formulated using antibodies that have an affinity for CCL23 splice variant, that is within a factor of 2, and most preferably a factor of 0.5 or less, relative to an affinity for CCL23. Alternatively, individual antibodies that separately bind CCL23 splice variant or CCL23 may be combined, either in a single assay, or in separate assays in which the assay results are combined computationally.

The CCL23 assays of the present invention may be used individually in a univariate fashion, or together with additional markers in a multivariate “panel” approach for diagnosis and/or prognosis. Such panels comprise measuring at least one, preferably at least two, more preferably at least three, still more preferably at least four, yet more preferably at least five, and most preferably at least six or more additional markers. These additional markers that may be used together with CCL23 assays of the present invention are described herein, and are preferably selected from the group consisting of markers related to blood pressure regulation, markers related to coagulation and hemostasis, markers related to apoptosis, and/or markers related to inflammation.

In certain preferred embodiments, the methods comprise one or more CCL23 assays of the present invention; and performing one or more additional immunoassays that detect markers selected from the group consisting of NT-proBNP, proBNP, BNP₇₉₋₁₀₈, BNP, BNP₃₋₁₀₈, CCL23, CRP, D-dimer, IL-Ira, NGAL, peptidoglycan recognition protein, procalcitonin, procalcitonin₃₋₁₁₆, active protein C, latent protein C, total protein C, and sTNFR1a to provide one or more additional immunoassay results.

In this “panel” approach, the relating step comprises relating the CCL23 assay result(s) obtained, and the one or more additional immunoassay results obtained, (I) to one or more of the following diagnoses: (i) the presence or absence of SIRS, (ii) the presence or absence of sepsis, (iii) the presence or absence of severe sepsis, and (iv) the presence or absence of septic shock; (2) to ruling in or out one or more of the following diagnoses: that the subject has SIRS, but not sepsis, severe sepsis, or septic shock; that the subject has sepsis, but not severe sepsis or septic shock; or that the subject has septic shock; (3) to the likelihood of a future outcome, either positive (e.g., that the subject is more likely to live, or is at a decreased risk of progressing to a more severe SIRS category) or negative (e.g., that the subject is at an increased risk of death, that the subject is at an increased risk of progressing to a more severe SIRS category); and/or (4) the success or failure of the treatment received. While exemplary panels are described herein, one or more markers may be replaced, added, or subtracted from these exemplary panels while still providing clinically useful results.

In certain embodiments, the relating step comprises comparing the concentrations of the individual marker(s) to one or more preselected levels (a “threshold”). Thresholds may be selected that provide an acceptable ability to predict diagnosis, prognostic risk, treatment success, etc. In practice, Receiver Operating Characteristic curves, or “ROC” curves, are typically calculated by plotting the value of a variable versus its relative frequency in two populations (called arbitrarily “disease” and “normal” or “low risk” and “high risk” for example). For any particular marker, a distribution of marker levels for subjects with and without a disease will likely overlap. Under such conditions, a test does not absolutely distinguish “disease” and “normal” with 100% accuracy, and the area of overlap indicates where the test cannot distinguish “disease” and “normal.” A threshold is selected, above which (or below which, depending on how a marker changes with the disease or prognosis) the test is considered to be “positive” and below which the test is considered to be “negative.” The area under the ROC curve is a measure of the probability that the perceived measurement will allow correct identification of a condition. See, e.g., Hanley et al., Radiology 143: 29-36 (1982).

Additionally, thresholds may be established by obtaining an earlier marker result from the same patient, to which later results may be compared. In these embodiments, the individual in effect acts as their own “control group.” In markers that increase with disease severity or prognostic risk, an increase over time in the same patient can indicate a worsening of disease or a failure of a treatment regimen, while a decrease over time can indicate remission of disease or success of a treatment regimen.

In certain embodiments, markers and/or marker panels are selected to distinguish “disease” and “normal” or, alternatively “low risk” from “high risk” with at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of a test's ability to predict disease, prognostic risk, or treatment outcome. In the case of a positive likelihood ratio, a value of 1 indicates that a positive result is equally likely among subjects in both the first and second groups; a value greater than 1 indicates that a positive result is more likely in the first group; and a value less than 1 indicates that a positive result is more likely in the second group. In the case of a negative likelihood ratio, a value of 1 indicates that a negative result is equally likely among subjects in both groups; a value greater than 1 indicates that a negative result is more likely in the first group; and a value less than 1 indicates that a negative result is more likely in the second group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the first and second groups; a value greater than 1 indicates that a positive result is more likely in the first group; and a value less than 1 indicates that a positive result is more likely in the second group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk is equal in both the first and second groups; a value greater than 1 indicates that the risk is greater in the first group; and a value less than 1 indicates that the risk is greater in the second group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

In some cases, multiple thresholds may be determined. This is the case in so-called “tertile,” “quartile,” or “quintile” analyses. In these methods, the “disease” and “normal” groups (or “low risk” and “high risk”) groups are considered together as a single population, and are divided into 3, 4, or 5 (or more) “bins” having equal numbers of individuals. The boundary between two of these “bins” may be considered “thresholds.” A risk (of a particular diagnosis or prognosis for example) can be assigned based on which “bin” a test subject falls into.

In other embodiments, particular thresholds for the marker(s) measured are not relied upon to determine if the marker level(s) obtained from a subject are correlated to a particular diagnosis or prognosis. For example, a temporal change in the marker(s) can be used to rule in or out one or more particular diagnoses and/or prognoses. Alternatively, marker(s) are correlated to a condition, disease, prognosis, etc., by the presence or absence of the marker(s) in a particular assay format. And in the case of panels, the present invention may utilize an evaluation of the entire profile of markers to provide a single result value (e.g., a “panel response” value expressed either as a numeric score or as a percentage risk). In such embodiments, an increase, decrease, or other change (e.g., slope over time) in a certain subset of markers may be sufficient to indicate a particular condition or future outcome in one patient, while an increase, decrease, or other change in a different subset of markers may be sufficient to indicate the same or a different condition or outcome in another patient. Methods for performing such analyses are described hereinafter.

The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al., Radiology 143: 29-36 (1982).

In certain embodiments, markers and/or marker panels are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +1-5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

In certain embodiments, a CCL23sv assay result, total CCL23 assay result, or both, is related to a diagnosis, and the relating step comprises comparing the assay result(s) to predetermined threshold(s) selected to provide a ROC area of at least 0.7 for the diagnosis of sepsis. In alternative embodiments, the CCL23sv immunoassay result is related to a diagnosis of severe sepsis, and the relating step comprises comparing the CCL23sv immunoassay result to a predetermined level of CCL23sv selected to provide a ROC area of at least 0.7 for the diagnosis of severe sepsis. In other embodiments, the CCL23sv immunoassay result is related to a diagnosis of septic shock, and the relating step comprises comparing the CCL23sv immunoassay result to a predetermined level of CCL23sv selected to provide a ROC area of at least 0.7 for the diagnosis of septic shock. In still other embodiments, the CCL23sv immunoassay result is related to a diagnosis of advanced sepsis, and the relating step comprises comparing the CCL23sv immunoassay result to a predetermined level of CCL23sv selected to provide a ROC area of at least 0.7 for the diagnosis of advanced sepsis.

In certain other embodiments, a CCL23sv assay result, total CCL23 assay result, or both, is related a prognosis of near-term mortality, and the relating step comprises comparing the assay result(s) to predetermined threshold(s) selected to provide an odds ratio of at least 2 for the prognostic risk of mortality. Such near-term mortality is death within 7 days, more preferably within 5 days, still more preferably within 3 days, and most preferably within 48 hours. Subjects for whom a prognostic risk is assigned may suffer from SIRS, sepsis, severe sepsis, septic shock or MODS. In certain preferred embodiments, the subject for whom a prognostic risk is assigned suffers from “advanced sepsis.”

In certain other embodiments, a CCL23sv assay result, total CCL23 assay result, or both, is related a prognosis of progressing to a worsening sepsis category, and the relating step comprises comparing the assay result(s) to predetermined threshold(s) selected to provide an odds ratio of at least 2 for the prognostic risk of progressing to a worsening sepsis category. Such risk is assigned for progressing to a worsening sepsis category within 7 days, more preferably within 5 days, still more preferably within 3 days, and most preferably within 48 hours. Subjects for whom a prognostic risk is assigned may suffer from SIRS, sepsis, severe sepsis, septic shock or MODS. In certain preferred embodiments, the subject for whom a prognostic risk is assigned suffers from “advanced sepsis.”

In another aspect, the invention relates to a method of formulating a total CCL23 assay, wherein the total CCL23 assay is insensitive for CCL23 splice variant, relative to CCL23.

In certain embodiments, these methods comprise providing at least two antibody populations that bind to an epitope that is present in both CCL23 splice variant and CCL23, and that pair with one another in a sandwich immunoassay format for detection of CCL23 splice variant and CCL23. Such assays may preferably be formulated using antibodies that have an affinity for CCL23 splice variant that is within a factor of 2, and most preferably a factor of 0.5 or less, relative to an affinity for CCL23. In preferred embodiments, one of these antibody populations is detectably labeled, and the other antibody population is attached to a solid phase.

In related embodiments, total CCL23 assays may be formulated by providing separate antibody populations, one of which binds to CCL23 splice variant, and the other of which binds CCL23. These separate antibody populations may be pooled to provide a pooled antibody that acts as if it a single antibody population that binds both CCL23 splice variant and CCL23. That pooled antibody can be used in a sandwich immunoassay format, either with an antibody population that binds to an epitope that is present in both CCL23 splice variant and CCL23 and that pairs with the pooled antibody in a sandwich immunoassay format for detection of CCL23 splice variant and CCL2, or with a second pooled antibody population formed in a similar manner. Again, in preferred embodiments, one of these antibody populations is detectably labeled, and the other antibody population is attached to a solid phase.

Alternatively, separate antibody populations, one of which binds to CCL23 splice variant, and the other of which binds CCL23, may be used in separate assays, one of which is a CCL23sv immunoassay, and the other of which is a CCL23-specific assay. The results are then combined computationally (e.g., by summing the concentrations of CCL23 splice variant and CCL23 obtained from these separate assays) to provide a total CCL23 assay result. Such assays are preferably sandwich assays in which one antibody is detectably labeled, and the other antibody is attached to a solid phase.

In a related aspect, the invention relates to devices to perform one or more of the methods described herein, and methods of their use. Such devices preferably contain a plurality of diagnostic zones, each of which is related to a particular marker of interest. Such diagnostic zones are preferably discrete locations within a single assay device. Such devices may be referred to as “arrays” or “microarrays.” Following reaction of a sample with the devices, a signal is generated from the diagnostic zone(s), which may then be correlated to the presence or amount of the markers of interest. Numerous suitable devices are known to those of skill in the art.

Such assay devices are preferably configured to provide reagents for performing the total CCL23 assays described above. In these embodiments, one or more assay zones comprise one or more solid phase antibodies as described in the preceeding paragraphs, and the assay device further comprises one or more detectably labeled antibodies as described in the preceeding paragraphs. Upon addition of a sample to the device, one or more sandwich assays are performed, from which a total CCL23 assay result is obtained. As noted above, such devices may perform a single assay from which the total CCL23 assay result is obtained, or the total result may be obtained from separate assays, one of which is a CCL23sv immunoassay, and the other of which is a CCL23-specific assay, the results of which are then combined computationally. Most preferably, the total CCL23 assay performed by the device is insensitive for CCL23 splice variant, relative to CCL23.

### DETAILED DESCRIPTION OF THE INVENTION

The present invention relates to methods and compositions for symptom-based differential diagnosis, prognosis, and determination of treatment regimens in subjects. In particular, the invention relates to methods and compositions selected to rule in or out SIRS, or for differentiating sepsis, severe sepsis, septic shock, and/or MODS from each other and/or from non-infectious SIRS.

Patients presenting for medical treatment often exhibit one or a few primary observable changes in bodily characteristics or functions that are indicative of disease. Often, these “symptoms” are nonspecific, in that a number of potential diseases can present the same observable symptom or symptoms. In the case of SIRS, the condition exists, by definition, whenever two or more of the following symptoms are present:

a temperature>38° C. or <36° C.;
a heart rate of >90 beats per minute (tachycardia);
a respiratory rate of >20 breaths per minute (tachypnea) or a PₐCO₂<4.3 kPa; and
a white blood cell count>12,000 per mm³, <4,000 per mm³, or >10% immature (band) forms.

The present invention describes methods and compositions that can assist in the differential diagnosis of one or more nonspecific symptoms by providing diagnostic markers that are designed to rule in or out one, and preferably a plurality, of possible etiologies for the observed symptoms. Symptom-based differential diagnosis described herein can be achieved using panels of diagnostic markers designed to distinguish between possible diseases that underlie a nonspecific symptom observed in a patient.

### DEFINITIONS

The term “CCL23 splice variant” as used herein refers to a mature polypeptide formed by removal of the signal sequence from the polypeptide described in Swiss-Prot accession number P55773-2. CCL23 splice variant has the following sequence:

```
(SEQ ID NO:1)
                10 20 30 40 50 60
RVTKDAETEF MMSKLPLENP VLLDMLWRRK IGPQMTLSHA AGFHATSADC CISYTPRSIP
                70 80 90 100 110 116
CSLLESYFET NSECSKPGVI FLTKKGRRFC ANPSDKQVQV CMRMLKLDTR IKTRKN.
```

The term “CCL23” as used herein refers to a mature polypeptide formed by removal of the signal sequence from the polypeptide described in Swiss-Prot accession number P55773-1. CCL23 has the following sequence:

```
(SEQ ID NO:2)
                10 20 30 40 50 60
RVTKDAETEF MMSKLPLENP VLLDRFHATS ADCCISYTPR SIPCSLLESY FETNSECSKP
                70 80 90 99
GVIFLTKKGR RFCANPSDKQ VQVCMRMLKL DTRIKTRKN.
```

As is apparent from these sequences, CCL23 splice variant is a longer variant of CCL23, in which R46 is replaced by MLWRRKIGPQMTLSHAAG (SEQ ID NO:3). In the case of both CCL23 splice variant and CCL23, the putative secretory signal sequence is represented by residues 1-21 (MKVSVAALSCLMLVTALGSQA, SEQ ID NO: 4), which are presumably lacking from the mature secreted form of each protein.

It has been reported that CCL23—the short form—is the major species and the longer CCL23 splice variant form was detected only in very low abundance. The present invention demonstrates that, in conditions related to SIRS, substantial concentrations of the CCL23 splice variant form can be detected and related to both diagnosis and prognosis, and measurement of this form, or of total CCL23 (meaning both CCL23 and CCL23 splice variant) can provide improved results, relative to measuring CCL23 itself.

Preferred assays are “configured to detect” a particular marker, in this case preferably CCL23 splice variant. Because an antibody epitope is on the order of 8 amino acids, an immunoassay will detect other polypeptides (e.g., related markers) so long as the other polypeptides contain the epitope(s) necessary to bind to the antibody used in the assay. Such other polypeptides are referred to as being “immunologically detectable” in the assay, and would include various isoforms. That an assay is “configured to detect” a marker means that an assay can generate a detectable signal indicative of the presence or amount of a physiologically relevant concentration of a particular marker of interest.

Such an assay may, but need not, specifically detect a particular marker (i.e., detect a marker but not some or all related markers). Thus, an assay that is configured to detect CCL23 splice variant could also detect CCL23 if the antibody used in such an assay recognize an epitope common to both forms. Alternatively, an antibody that recognizes an epitope that is present in CCL23 splice variant but not CCL23 could be used to provide a CCL23sv immunoassay, and an antibody that binds to an epitope that is present in CCL23 but not CCL23 splice variant (such as an epitope formed by the junction around residue R46 in CCL23) could be used to provide a CCL23-specific immunoassay.

Additionally, N-terminal processed forms of CCL23, including CCL23₁₉₋₉₉, CCL23₂₂₋₉₉, CCL23₂₇₋₉₉, and CCL23₃₀₉₉, have been reported to be found in high levels in synovial fluids from rheumatoid patients. Because these N-terminal cleavages lie before the insertion at R46, an assay that is configured to detect CCL23 splice variant could also detect corresponding N-terminal processed forms of the splice variant.

Immunoassays may be configured in a variety of formats known in the art. In the case of a competitive immunoassay, markers to be detected must contain the epitope bound by the single antibody used in the assay in order to be detected. In the case of a sandwich immunoassay, markers to be detected must contain at least two epitopes bound by the antibody used in the assay in order to be detected. Taking CCL23 splice variant as an example, an assay configured to detect this marker may be configured to be a “total” CCL23 assay by selecting antibodies that bind in the regions that are common to both CCL23 and CCL23 splice variant. Alternatively, an assay may be configured to be specific to CCL23 splice variant, relative to CCL23, by selecting at least one antibody that binds to the splice variant but not to CCL23. It should be recognized that, in a sandwich assay that is specific to CCL23 splice variant relative to CCL23, only one antibody of the antibody pair used needs to be specific for the splice variant, as a signal is only obtained when both antibodies bind to the target polypeptide.

Preferred CCL23 splice variant assays may be described herein as being “sensitive” or “insensitive” for CCL23 splice variant, relative to CCL23. An “insensitive” assay as that term is used with regard to a target molecule is configured to provide a signal that is within a factor of 5, more preferably within a factor of two, and most preferably within 20%, when comparing assay results for equimolar amounts of the target and non-target. A “sensitive” assay as that term is used with regard to a target molecule is configured to provide a signal that is at least a factor of 5, more preferably a factor of ten, and most preferably a factor of 100 or more, greater when comparing assay results for equimolar amounts of the target and non-target. Certain CCL23 splice variant assays are sensitive, relative to CCL23. Particularly preferred CCL23 splice variant assays are insensitive relative to CCL23, and may also optionally bind one or more N-terminal processed forms of CCL23 selected from the group consisting of CCL23₁₉₋₉₉, CCL23₂₂₋₉₉, CCL23₂₇₋₉₉, and CCL23₃₀₋₉₉.

The term “antibody” as used herein refers to a peptide or polypeptide, or a population of peptides or polypeptides, derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Preferably the affinity of the antibody in a “sensitive” assay will be at least about 5 fold, preferably 10 fold, more preferably 25-fold, even more preferably 50-fold, and most preferably 100-fold or more, greater for a target molecule than its affinity for a non-target molecule.

The term “specifically binds” is not intended to indicate that an antibody binds exclusively to its intended target. Rather, an antibody “specifically binds” if its affinity for its intended target is about 5-fold greater when compared to its affinity for a non-target molecule. In preferred embodiments, Specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

Certain immunoassays of the present invention utilize at least one antibody that specifically binds CCL23 splice variant (the “target”), relative to CCL23 (the “non-target”), while certain other immunoassays of the present invention utilize antibody that binds both CCL23 and CCL23 splice variant with affinities that are within a factor of 5, and most preferably within a factor of 2 or less.

The term “marker” as used herein refers to proteins, polypeptides, glycoproteins, proteoglycans, lipids, lipoproteins, glycolipids, phospholipids, nucleic acids, carbohydrates, etc. or small molecules to be used as targets for screening test samples obtained from subjects. “Proteins or polypeptides” used as markers in the present invention are contemplated to include any fragments thereof, in particular, immunologically detectable fragments. Markers may post-translationally modified, for example by oxidation of methionine residues, ubiquitination, cysteinylation, nitrosylation (e.g., containing nitrotyrosine residues), halogenation (e.g., containing chlorotyrosine and/or bromotyrosine residues), glycosylation, complex formation, differential splicing, etc. Markers can also include clinical “scores” such as a pre-test probability assignment, a pulmonary hypertension “Daniel” score, an NIH stroke score, a Sepsis Score of Elebute and Stoner, a Duke Criteria for Infective Endocarditis, a Mannheim Peritonitis Index, an “Apache” score, etc.

Preferably, the methods described hereinafter utilize one or more markers that are derived from the subject. The term “subject-derived marker” as used herein refers to protein, polypeptide, phospholipid, nucleic acid, prion, glycoprotein, proteoglycan, glycolipid, lipid, lipoprotein, carbohydrate, or small molecule markers that are expressed or produced by one or more cells of the subject. The presence, absence, amount, or change in amount of one or more markers may indicate that a particular disease is present, or may indicate that a particular disease is absent. Additional markers may be used that are derived not from the subject, but rather that are expressed by pathogenic or infectious organisms that are correlated with a particular disease. Such markers are preferably protein, polypeptide, phospholipid, nucleic acid, prion, or small molecule markers that identify the infectious diseases described above. CCL23 splice variant and CCL23 are each subject-derived markers.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

As used herein, a “plurality” as used herein refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are “patients,” i.e., living humans that are receiving medical care for a disease or condition. This includes persons with no defined illness who are being investigated for signs of pathology.

The term “diagnosis” as used herein refers to methods by which the skilled artisan can estimate and/or determine whether or not a patient is suffering from a given disease or condition. It thus refers to a relative probability that a certain disease is present in the subject, and not the ability of a “specific marker” to give a definitive yes/no answer to the existence of a disease. The skilled artisan often makes a diagnosis on the basis of one or more diagnostic indicators, i.e., a marker, the presence, absence, amount, or change in amount of which is indicative of the presence, severity, or absence of the condition. The term “diagnosis” is not meant to refer to assigning the presence or absence of a particular disease or condition with absolute certainty, or even that a particular disease or condition is more likely than not. Rather, one or more markers may be used to indicate an increased or decreased risk of a particular disease or condition. For example, if CCL23 is increased above a particular level, that may indicate an increased likelihood that the subject under study suffers from sepsis, relative to a subject having a lower CCL23 level.

Similarly, the term “prognosis” refers to a relative probability that a certain future outcome will occur in the subject, and not the ability of a “specific marker” to give a definitive yes/no answer to the future outcome. A prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability for experiencing a future stroke in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient. Preferred prognostic markers can predict the chance of mortality in the “near term,” which as used herein refers to risk within 7 days of obtaining the sample in which the prognostic indicator is measured.

The term “correlating” or “relating” as used herein in reference to the use of markers refers to comparing the presence or amount of the marker(s) in a patient to its presence or amount in persons known to suffer from, or known to be at risk of, a given condition, or in persons known to be free of a given condition, and assigning an increased or decreased probability of a particular diagnosis, prognosis, etc., to an individual based on the assay result(s) obtained from that individual. Relating an assay result to the presence or absence of a particular disease or prognosis is not meant to indicate that the assay result(s) will have a level of sensitivity and specificity that meets the ideal of 100%. Moreover, the artisan understands that markers need not be elevated in a single specific condition for such markers to be useful to the artisan in clinical diagnosis. Few, if any, such definitive tests exist.

In the simple example where an the CCL23 assays described herein are used in a univariate fashion, relating the assay results to a diagnosis or prognosis may mean comparing the measured assay result (e.g., CCL23 concentration) to a predetermined CCL23 threshold concentration arrived at by examining a population of “normal” and “diseased” subjects and selecting a threshold that provides an acceptable level of sensitivity and specificity, an acceptable odds ratio, etc. A greater probability of particular diagnosis, prognosis, etc., is assigned to the subject above the threshold, relative to that which would be assigned below the threshold. That probability may be measured qualitatively (e.g., the subject is at an increased risk of having a sepsis classification that is more severe than sepsis above the threshold than below the threshold”) or quantitatively (e.g., “the odds ratio for the subject having a sepsis classification that is more severe than sepsis is 5-fold higher above the threshold than below the threshold”). Alternatively, a “quartile” approach may be used, where the probability of particular diagnosis, prognosis, etc. is assigned based on into which bin of the quartile the measured assay result falls. Numerous other ways to express the relationship of the assay results to a diagnosis or prognosis are known in the art.

A marker level in a subject's sample can be compared to a level known to be associated with a particular diagnosis. The sample's marker level is said to have been correlated with a diagnosis; that is, the skilled artisan can use the marker level to determine whether the patient likely suffers from a specific type diagnosis, and respond accordingly. Alternatively, the sample's marker level can be compared to a marker level known to be associated with a good outcome (e.g., a decreased likelihood of progressing to a more severe sepsis classification, etc.) in a “rule out” approach. In preferred embodiments, a profile of marker levels are correlated to a global probability or a particular outcome using ROC curves.

The term “discrete” as used herein refers to areas of a surface that are non-contiguous. That is, two areas are discrete from one another if a border that is not part of either area completely surrounds each of the two areas.

The term “independently addressable” as used herein refers to discrete areas of a surface from which a specific signal may be obtained.

The term “appreciable” as used herein with regard to assay signals and assay results, refers to a signal or result that is above background for a physiologically relevant concentration of an analyte.

The term “physiologically relevant concentration” as used herein refers to the average concentration of an analyte naturally present in a non-diseased subject population.

The term “therapy regimen” refers to one or more interventions made by a caregiver in hopes of treating a disease or condition. Therapy regimens for sepsis are well known in the art. Included is the “early sepsis therapy regimen,” which as used herein refers to a set of supportive therapies designed to reduce the risk of mortality when administered within the initial 24 hours, more preferably within the initial 12 hours, and most preferably within the initial 6 hours or earlier, of assigning a diagnosis of SIRS, sepsis, severe sepsis, septic shock, or MODS to a subject. Such supportive therapies comprise a spectrum of treatments including resuscitation, fluid delivery, vasopressor administration, inotrope administration, steroid administration, blood product administration, and/or sedation. See, e.g., Dellinger et al., Crit. Care Med. 32: 858-873, 2004, and Rivers et al., N. Engl. J. Med. 345: 1368-1377, 2001 (providing a description of “early goal directed therapy” as that term is used herein), each of which is hereby incorporated by reference. Preferably, such an early sepsis therapy regimen comprises one or more, and preferably a plurality, of the following therapies:

maintenance of a central venous pressure of 8-12 mm Hg, preferably by administration of crystalloids and/or colloids as necessary;
maintenance of a mean arterial pressure of >65 mm Hg, preferably by administration of vasopressors and/or vasodilators as necessary;
maintenance of a central venous oxygen saturation of >70%, preferably by administration of transfused red blood cells to a hematocrit of at least 30% and/or administration of dobutamine as necessary; and
administration of mechanical ventilation as necessary.

The term “related marker” as used herein refers to one or more immunologically detectable fragments of a particular marker or its biosynthetic parent that comprise 8 or more contiguous residues of the marker or its parent.

For example, human BNP is derived by proteolysis of a 108 amino acid precursor molecule, referred to hereinafter as BNP₁₋₁₀₈. Mature BNP, or “the BNP natriuretic peptide,” or “BNP-32” is a 32 amino acid molecule representing amino acids 77-108 of this precursor, which may be referred to as BNP₇₇₋₁₀₈. The remaining residues I-76 are referred to hereinafter as BNP₁₋₇₆, and are also known as “NT-proBNP.”

The sequence of the 108 amino acid BNP precursor pro-BNP (BNP₁₋₁₀₈) is as follows, with mature BNP (BNP₇₇₋₁₀₈) underlined:

| (SEQ ID NO: 5) | |
|---|---|
| HPLGSPGSAS DLETSGLQEQ RNHLQGKLSE LQVEQTSLEP LQESPRPTGV | 50 |
| WKSREVATEG IRGHRKMVLY TLRAPRSPKM1VQGSGCFGRK MDRISSSSGL | 100 |
| GCKVLRRH. | 108 |

BNP₁₋₁₀₈ is synthesized as a larger precursor pre-pro-BNP having the following sequence (with the “pre” sequence shown in bold):

| (SEQ ID NO: 6) | |
|---|---|
| MDPQTAPSRA LLLLLFLHLA FLGGRSHPLG SPGSASDLET SGLQEQRNHL | 50 |
| QGKLSELQVE QTSLEPLQES PRPTGVWKSR EVATEGIRGH RKMVLYTLRA | 100 |
| PRSPKMVQGS GCFGRKMDRI SSSSGLGCKV LRRH. | 134 |

While mature BNP itself may be used as a marker in the present invention, the prepro-BNP, BNP₁₋₁₀₈ and BNP₁₋₇₆ molecules represent BNP-related markers that may be measured either as surrogates for mature BNP or as markers in and of themselves. In addition, one or more fragments of these molecules, including BNP-related polypeptides selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈ may also be present in circulation. In addition, natriuretic peptide fragments, including BNP fragments, may comprise one or more oxidizable methionines, the oxidation of which to methionine sulfoxide or methionine sulfone produces additional BNP-related markers. See, e.g., U.S. patent Ser. No. 10/419,059, filed Apr. 17, 2003, which is hereby incorporated by reference in its entirety including all tables, figures and claims.

Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result. In addition, individual antibodies that distinguish amongst a plurality of marker fragments may be individually employed to separately detect the presence or amount of different fragments. The results of this individual detection may provide a more accurate prognostic or diagnostic result than detecting the plurality of fragments in a single assay. For example, different weighting factors may be applied to the various fragment measurements to provide a more accurate estimate of the amount of natriuretic peptide originally present in the sample.

In a similar fashion, many of the markers described herein are synthesized as larger precursor molecules, which are then processed to provide mature marker; and/or are present in circulation in the form of fragments of the marker. Thus, “related markers” to each of the markers described herein may be identified and used in an analogous fashion to that described above for BNP.

Removal of polypeptide markers from the circulation often involves degradation pathways. Moreover, inhibitors of such degradation pathways may hold promise in treatment of certain diseases. See, e.g., Trindade and Rouleau, Heart Fail. Monit. 2: 2-7, 2001. However, the measurement of the polypeptide markers has focused generally upon measurement of the intact form without consideration of the degradation state of the molecules. Assays may be designed with an understanding of the degradation pathways of the polypeptide markers and the products formed during this degradation, in order to accurately measure the biologically active forms of a particular polypeptide marker in a sample. The unintended measurement of both the biologically active polypeptide marker(s) of interest and inactive fragments derived from the markers may result in an overestimation of the concentration of biologically active form(s) in a sample.

The failure to consider the degradation fragments that may be present in a clinical sample may have serious consequences for the accuracy of any diagnostic or prognostic method. Consider for example a simple case, where a sandwich immunoassay is provided for BNP, and a significant amount (e.g., 50%) of the biologically active BNP that had been present has now been degraded into an inactive form. An immunoassay formulated with antibodies that bind a region common to the biologically active BNP and the inactive fragment(s) will overestimate the amount of biologically active BNP present in the sample by 2-fold, potentially resulting in a “false positive” result. Overestimation of the biologically active form(s) present in a sample may also have serious consequences for patient management. Considering the BNP example again, the BNP concentration may be used to determine if therapy is effective (e.g., by monitoring BNP to see if an elevated level is returning to normal upon treatment). The same “false positive” BNP result discussed above may lead the physician to continue, increase, or modify treatment because of the false impression that current therapy is ineffective.

Likewise, it may be necessary to consider the complex state of one or more markers described herein. For example, troponin exists in muscle mainly as a “ternary complex” comprising three troponin polypeptides (T, I and C). But troponin I and troponin T circulate in the blood in forms other than the I/T/C ternery complex. Rather, each of (i) free cardiac-specific troponin I, (ii) binary complexes (e.g., troponin I/C complex), and (iii) ternary complexes all circulate in the blood. Furthermore, the “complex state” of troponin I and T may change over time in a patient, e.g., due to binding of free troponin polypeptides to other circulating troponin polypeptides. Immunoassays that fail to consider the “complex state” of troponin may not detect all of the cardiac-specific isoform of interest.

### Selecting a Threshold

The artisan understands that even for biomarkers that are routinely used in the medical setting, the performance characteristics, such as the desired specificity and sensitivity, appropriate thresholds, etc., for the particular test and patient population under study must be established by the skilled artisan. While it may seem that two assays for a particular biomarker should give the same result (that is, that 100 ng/mL is 100 ng/mL, no matter what test is being used), that is not typically the case for immunoassays.

For example, in the case of cardiac troponin I (a marker of myocardial damage commonly assayed in clinical laboratories), it has been reported that measurements using different commercial FDA-approved troponin I assays on identical specimens may differ in measured concentration by 100-fold. See, e.g., Christenson et al., “Standardization of Cardiac Troponin I Assays: Round Robin of Ten Candidate Reference Materials,” Clin. Chem. 47: 431-37 (2001). Said another way, a threshold concentration selected for a particular assay platform may not translate to a different assay platform. Thus, in developing a particular marker test, the artisan understands that appropriate thresholds, the concentration of a particular marker in an individual, the concentration that is considered “physiologically relevant,” etc., need to be determined for that particular test, and certain well established methods are often used to do so.

In one embodiment, levels of the marker(s) being employed are obtained from a group of subjects that is divided into at least two sets. The first set includes subjects who have been confirmed as having a disease, outcome, or, more generally, being in a first condition state. For example, this first set of patients may be those diagnosed with severe sepsis (diagnosis group), those that progress to a worsening sepsis category (prognosis group), or those that improve following treatment (therapy group). Subjects in this first set will be referred to as “diseased,” however this label is arbitrary. The second set of subjects is simply those who do not fall within the first set. Subjects in this second set will be referred to as “non-diseased,” although again this label is arbitrary. Preferably, the first set and the second set each have an approximately equal number of subjects. The second set may be normal patients, and/or patients that do not suffer from recurrence, and/or that fail to improve or worsen following treatment.

In addition, serial testing of a marker in the same patient may also be used to establish a threshold. In effect, an earlier assay result from the same patient acts as a threshold to which later results may be compared. For example, procalcitonin (PCT) has been proposed as a marker of disease severity in sepsis, and serial measurements have been suggested to monitor response to therapy. Similarly, persistently high CRP concentrations have been associated with a poor outcome, and serial measurements may be used to identify those patients who require more aggressive interventions to prevent complications, and anti-inflammatory cytokine levels such as IL-1ra reportedly remain elevated in patients that suffer from multiple organ failure, while in patients without multiple organ failure such levels decline.

As noted above, a single marker often is incapable of definitively identifying a subject as falling within a first or second group. For example, if a patient is measured as having a marker level that falls within an overlapping region in the distribution of diseased and non-diseased subjects, the results of the test may be useless in diagnosing the patient. A cutoff may be established to distinguish between a positive and a negative test result for the detection of the disease or condition. Regardless of where the cutoff is selected, the effectiveness of the single marker as a diagnosis tool is unaffected. Changing the cutoff trades off between the number of false positives and the number of false negatives resulting from the use of the single marker.

The effectiveness of a test having such an overlap is often expressed using a ROC (Receiver Operating Characteristic) curve. ROC curves are well known to those skilled in the art. The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

Measures of test accuracy may be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, preferred tests and assays exhibit one or more of the following results on these various measures:

at least 70% sensitivity,
at least 70% specificity;
an odds ratio of at least 3 or 0.33;
ROC curve area of at least 0.6, more preferably 0.7, still more preferably at least 0.8, even more preferably at least 0.9, and most preferably at least 0.95; and/or
a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.
Use of CCL23 Assays in Combination with Other Clinical Indicia

Once obtained, the relationship of the assay results to a particular diagnosis or prognosis may be used in a variety of manners. For example, a diagnosis indicating an increased diagnostic or prognostic risk may result in sending the subject for additional diagnostic tests. An increased risk of a particular diagnosis or prognosis may be assigned to a subject based on the use of one or more CCL23 assays of the present invention by comparing a measured concentration to some cutoff. That risk may be further increased if another marker also indicates an increased risk of the same diagnosis or prognosis, or may be decreased if an another marker indicates a decreased risk of the same diagnosis or prognosis. As discussed herein, markers may include subject-derived markers, but may also include clinical indicia of a patient's disease state, such as the Acute Physiology and Chronic Health Evaluation II (APACHE II) score, Elebute score, Multiple Organ Failure-Goris score, Simplified Acute Physiology Score, Sepsis Severity Score, or Mannheim Peritonitis Index (MPI). This list is not meant to be limiting.

A panel consisting of the markers referenced herein and/or their related markers may be constructed to provide relevant information related to the diagnosis of interest. Such a panel may be constructed using 1, 2, 3, 4, 5, 6, 7, 8, 9, 10, 11, 12, 13, 14, 15, 16, 17, 18, 19, 20, or more individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity.

The following table provides a list of additional preferred markers for use in the present invention. Further detail is provided in US2005/0148029, which is hereby incorporated by reference in its entirety. As described herein, markers related to each of these markers are also encompassed by the present invention.

| Marker | Classification |
|---|---|
| Myoglobin | Tissue injury |
| E-selectin | Tissue injury |
| VEGF | Tissue injury |
| EG-VEGF | Tissue injury |
| Troponin I and complexes | Myocardial injury |
| Troponin T and complexes | Myocardial injury |
| Annexin V | Myocardial injury |
| B-enolase | Myocardial injury |
| CK-MB | Myocardial injury |
| Glycogen phosphorylase-BB | Myocardial injury |
| Heart type fatty acid binding protein | Myocardial injury |
| Phosphoglyceric acid mutase | Myocardial injury |
| S-100ao | Myocardial injury |
| ANP | Blood pressure regulation |
| CNP | Blood pressure regulation |
| Kininogen | Blood pressure regulation |
| CGRP II | Blood pressure regulation |
| urotensin II | Blood pressure regulation |
| BNP | Blood pressure regulation |
| NT-proBNP | Blood pressure regulation |
| proBNP | Blood pressure regulation |
| calcitonin gene related peptide | Blood pressure regulation |
| arg-Vasopressin | Blood pressure regulation |
| Endothelin-1 (and/or Big ET-1) | Blood pressure regulation |
| Endothelin-2 (and/or Big ET-2) | Blood pressure regulation |
| Endothelin-3 (and/or Big ET-3) | Blood pressure regulation |
| procalcitonin | Blood pressure regulation |
| calcyphosine | Blood pressure regulation |
| adrenomedullin | Blood pressure regulation |
| aldosterone | Blood pressure regulation |
| angiotensin 1 (and/or angiotensinogen 1) | Blood pressure regulation |
| angiotensin 2 (and/or angiotensinogen 2) | Blood pressure regulation |
| angiotensin 3 (and/or angiotensinogen 3) | Blood pressure regulation |
| Bradykinin | Blood pressure regulation |
| Tachykinin-3 | Blood pressure regulation |
| calcitonin | Blood pressure regulation |
| Renin | Blood pressure regulation |
| Urodilatin | Blood pressure regulation |
| Ghrelin | Blood pressure regulation |
| Plasmin | Coagulation and hemostasis |
| Thrombin | Coagulation and hemostasis |
| Antithrombin-III | Coagulation and hemostasis |
| Fibrinogen | Coagulation and hemostasis |
| von Willebrand factor | Coagulation and hemostasis |
| D-dimer | Coagulation and hemostasis |
| PAI-1 | Coagulation and hemostasis |
| Protein C | Coagulation and hemostasis |
| Soluble Endothelial Protein C Receptor | Coagulation and hemostasis |
| (EPCR) | |
| TAFI | Coagulation and hemostasis |
| Fibrinopeptide A | Coagulation and hemostasis |
| Plasmin alpha 2 antiplasmin complex | Coagulation and hemostasis |
| Platelet factor 4 | Coagulation and hemostasis |
| Platelet-derived growth factor | Coagulation and hemostasis |
| P-selectin | Coagulation and hemostasis |
| Prothrombin fragment 1 + 2 | Coagulation and hemostasis |
| B-thromboglobulin | Coagulation and hemostasis |
| Thrombin antithrombin III complex | Coagulation and hemostasis |
| Thrombomodulin | Coagulation and hemostasis |
| Thrombus Precursor Protein | Coagulation and hemostasis |
| Tissue factor | Coagulation and hemostasis |
| Tissue factor pathway inhibitor-α | Coagulation and hemostasis |
| Tissue factor pathway inhibitor-β | Coagulation and hemostasis |
| basic calponin 1 | Vascular tissue |
| beta like 1 integrin | Vascular tissue |
| Calponin | Vascular tissue |
| CSRP2 | Vascular tissue |
| elastin | Vascular tissue |
| Endothelial cell-selective adhesion molecule | Vascular tissue |
| (ESAM) | |
| Fibrillin 1 | Vascular tissue |
| Junction Adhesion Molecule-2 | Vascular tissue |
| LTBP4 | Vascular tissue |
| smooth muscle myosin | Vascular tissue |
| transgelin | Vascular tissue |
| Carboxyterminal propeptide of type I | Collagen synthesis |
| procollagen (PICP) | |
| Collagen carboxyterminal telopeptide (ICTP) | Collagen degradation |
| APRIL (TNF ligand superfamily member 13) | Inflammatory |
| CD27 (TNFRSF7) | Inflammatory |
| Complement C3a | Inflammatory |
| CCL-5 (RANTES) | Inflammatory |
| CCL-8 (MCP-2) | Inflammatory |
| CCL-16 | Inflammatory |
| CCL-19 (macrophage inflammatory protein- | Inflammatory |
| 3β) | |
| CCL-20 (MIP-3α) | Inflammatory |
| CCL-23 (MIP-3) | Inflammatory |
| CXCL-5 (small inducible cytokine B5) | Inflammatory |
| CXCL-9 (small inducible cytokine B9) | Inflammatory |
| CXCL-13 (small inducible cytokine B13) | Inflammatory |
| CXCL-16 (small inducible cytokine B16) | Inflammatory |
| DPP-II (dipeptidyl peptidase II) | Inflammatory |
| DPP-IV (dipeptidyl peptidase IV) | Inflammatory |
| Glutathione S Transferase | Inflammatory |
| HIF 1 ALPHA | Inflammatory |
| IL-25 | Inflammatory |
| IL-23 | Inflammatory |
| IL-22 | Inflammatory |
| IL-18 | Inflammatory |
| IL-13 | Inflammatory |
| IL-12 | Inflammatory |
| IL-10 | Inflammatory |
| IL-1-Beta | Inflammatory |
| IL-1ra | Inflammatory |
| IL-4 | Inflammatory |
| IL-6 | Inflammatory |
| IL-8 | Inflammatory |
| Lysophosphatidic acid | Inflammatory |
| MDA-modified LDL | Inflammatory |
| Human neutrophil elastase | Inflammatory |
| C-reactive protein | Inflammatory |
| Insulin-like growth factor | Inflammatory |
| Inducible nitric oxide synthase | Inflammatory |
| Intracellular adhesion molecule | Inflammatory |
| NGAL (Lipocalin-2) | Inflammatory |
| Lactate dehydrogenase | Inflammatory |
| MCP-1 | Inflammatory |
| MMP-1 | Inflammatory |
| MMP-2 | Inflammatory |
| MMP-3 | Inflammatory |
| MMP-7 | Inflammatory |
| MMP-9 | Inflammatory |
| TIMP-1 | Inflammatory |
| TIMP-2 | Inflammatory |
| TIMP-3 | Inflammatory |
| NGAL | Inflammatory |
| n-acetyl aspartate | Inflammatory |
| PTEN | Inflammatory |
| Phospholipase A2 | Inflammatory |
| TNF Receptor Superfamily Member 1A | Inflammatory |
| TNFRSF3 (lymphotoxin β receptor) | Inflammatory |
| Transforming growth factor beta | Inflammatory |
| TREM-1 | Inflammatory |
| TREM-1sv | Inflammatory |
| TL-1 (TNF ligand related molecule-1) | Inflammatory |
| TL-1a | Inflammatory |
| Tumor necrosis factor alpha | Inflammatory |
| Vascular cell adhesion molecule | Inflammatory |
| Vascular endothelial growth factor | Inflammatory |
| cystatin C | Inflammatory |
| substance P | Inflammatory |
| Myeloperoxidase (MPO) | Inflammatory |
| macrophage inhibitory factor | Inflammatory |
| Fibronectin | Inflammatory |
| cardiotrophin 1 | Inflammatory |
| Haptoglobin | Inflammatory |
| PAPPA | Inflammatory |
| s-CD40 ligand | Inflammatory |
| HMG-1 (or HMGB1) | Inflammatory |
| IL-2 | Inflammatory |
| IL-4 | Inflammatory |
| IL-11 | Inflammatory |
| IL-13 | Inflammatory |
| IL-18 | Inflammatory |
| Eosinophil cationic protein | Inflammatory |
| Mast cell tryptase | Inflammatory |
| VCAM | Inflammatory |
| sICAM-1 | Inflammatory |
| TNFα | Inflammatory |
| Osteoprotegerin | Inflammatory |
| Prostaglandin D-synthase | Inflammatory |
| Prostaglandin E2 | Inflammatory |
| RANK ligand | Inflammatory |
| RANK (TNFRSF11A) | Inflammatory |
| HSP-60 | Inflammatory |
| Serum Amyloid A | Inflammatory |
| s-iL 18 receptor | Inflammatory |
| S-iL-1 receptor | Inflammatory |
| s-TNF P55 | Inflammatory |
| s-TNF P75 | Inflammatory |
| sTLR-1 (soluble toll-like receptor-1) | Inflammatory |
| sTLR-2 | Inflammatory |
| sTLR-4 | Inflammatory |
| TGF-beta | Inflammatory |
| MMP-11 | Inflammatory |
| Beta NGF | Inflammatory |
| CD44 | Inflammatory |
| EGF | Inflammatory |
| E-selectin | Inflammatory |
| Fibronectin | Inflammatory |
| RAGE | Inflammatory |
| Neutrophil elastase | Pulmonary injury |
| KL-6 | Pulmonary injury |
| LAMP 3 | Pulmonary injury |
| LAMP3 | Pulmonary injury |
| Lung Surfactant protein A | Pulmonary injury |
| Lung Surfactant protein B | Pulmonary injury |
| Lung Surfactant protein C | Pulmonary injury |
| Lung Surfactant protein D | Pulmonary injury |
| phospholipase D | Pulmonary injury |
| PLA2G5 | Pulmonary injury |
| SFTPC | Pulmonary injury |
| MAPK10 | Neural tissue injury |
| KCNK4 | Neural tissue injury |
| KCNK9 | Neural tissue injury |
| KCNQ5 | Neural tissue injury |
| 14-3-3 | Neural tissue injury |
| 4.1B | Neural tissue injury |
| APO E4-1 | Neural tissue injury |
| myelin basic protein | Neural tissue injury |
| Atrophin 1 | Neural tissue injury |
| Brain derived neurotrophic factor | Neural tissue injury |
| Brain fatty acid binding protein | Neural tissue injury |
| Brain tubulin | Neural tissue injury |
| CACNA1A | Neural tissue injury |
| Calbindin D | Neural tissue injury |
| Calbrain | Neural tissue injury |
| Carbonic anhydrase XI | Neural tissue injury |
| CBLN1 | Neural tissue injury |
| Cerebellin 1 | Neural tissue injury |
| Chimerin 1 | Neural tissue injury |
| Chimerin 2 | Neural tissue injury |
| CHN1 | Neural tissue injury |
| CHN2 | Neural tissue injury |
| Ciliary neurotrophic factor | Neural tissue injury |
| CK-BB | Neural tissue injury |
| CRHR1 | Neural tissue injury |
| C-tau | Neural tissue injury |
| DRPLA | Neural tissue injury |
| GFAP | Neural tissue injury |
| GPM6B | Neural tissue injury |
| GPR7 | Neural tissue injury |
| GPR8 | Neural tissue injury |
| GRIN2C | Neural tissue injury |
| GRM7 | Neural tissue injury |
| HAPIP | Neural tissue injury |
| HIP2 | Neural tissue injury |
| LDH | Neural tissue injury |
| Myelin basic protein | Neural tissue injury |
| NCAM | Neural tissue injury |
| NT-3 | Neural tissue injury |
| NDPKA | Neural tissue injury |
| Neural cell adhesion molecule | Neural tissue injury |
| NEUROD2 | Neural tissue injury |
| Neurofiliment L | Neural tissue injury |
| Neuroglobin | Neural tissue injury |
| neuromodulin | Neural tissue injury |
| Neuron specific enolase | Neural tissue injury |
| Neuropeptide Y | Neural tissue injury |
| Neurotensin | Neural tissue injury |
| Neurotrophin 1, 2, 3, 4 | Neural tissue injury |
| NRG2 | Neural tissue injury |
| PACE4 | Neural tissue injury |
| phosphoglycerate mutase | Neural tissue injury |
| PKC gamma | Neural tissue injury |
| proteolipid protein | Neural tissue injury |
| PTEN | Neural tissue injury |
| PTPRZ1 | Neural tissue injury |
| RGS9 | Neural tissue injury |
| RNA Binding protein Regulatory Subunit | Neural tissue injury |
| S-100β | Neural tissue injury |
| SCA7 | Neural tissue injury |
| secretagogin | Neural tissue injury |
| SLC1A3 | Neural tissue injury |
| SORL1 | Neural tissue injury |
| SREB3 | Neural tissue injury |
| STAC | Neural tissue injury |
| STX1A | Neural tissue injury |
| STXBP1 | Neural tissue injury |
| Syntaxin | Neural tissue injury |
| thrombomodulin | Neural tissue injury |
| transthyretin | Neural tissue injury |
| adenylate kinase-1 | Neural tissue injury |
| BDNF | Neural tissue injury |
| neurokinin A | Neural tissue injury |
| neurokinin B | Neural tissue injury |
| s-acetyl Glutathione | apoptosis |
| cytochrome C | apoptosis |
| Caspase 3 | apoptosis |
| Cathepsin D | apoptosis |
| α-spectrin | apoptosis |

Preferred panels comprise combining a CCL23 splice variant assay and/or total CCL23 assay with one or more additional immunoassays that detect markers selected from the group consisting of NT-proBNP, proBNP, BNP₇₉₋₁₀₈, BNP, BNP₃₋₁₀₈, CCL23 (CCL23-specific), CRP, D-dimer, IL-Ira, NGAL, peptidoglycan recognition protein, procalcitonin, procalcitonin₃₋₁₁₆, active protein C, latent protein C, total protein C, and sTNFR1a. Particularly preferred panels comprise combining a CCL23 splice variant assay and/or total CCL23 assay with one or more of a CCL23-specific assay, an NGAL assay, and a CRP assay. Most preferred panels comprise combining a CCL23 splice variant assay and/or total CCL23 assay with an NGAL assay, and a CRP assay.

One skilled in the art will also recognize that univariate analysis of markers can be performed and the data from the univariate analyses of multiple markers can be combined to form panels of markers to differentiate different disease conditions. Such methods include multiple linear regression, determining interaction terms, stepwise regression, etc. In preferred embodiments, marker panels combine multiple marker assay results into a single composite result. This single composite result may be used as if it is a single marker, and so subjected to ROC analysis to select decision thresholds, etc. Suitable methods for identifying and using markers panels are described in detail in U.S. Provisional Patent Application No. 60/436,392 filed Dec. 24, 2002, PCT application US03/41426 filed Dec. 23, 2003, U.S. patent application Ser. No. 10/331,127 filed Dec. 27, 2002, and PCT application No. US03/41453, each of which is hereby incorporated by reference in its entirety, including all tables, figures, and claims.

Clinical and marker data may also be combined using classification trees (also known as decision trees). Many statistical software packages are available that will implement this given the clinical data in the format X(m,n) and R(n). For example, MATLAB, or CART, or SPSS, etc. The trees may be produced with a large variety of splitting rules, prior probabilities, and weighting schemes. The trees may be fit to an arbitrary level of detail, or pruned using various cross-validation methods to avoid over-fitting the data. Large ensembles of trees may also be combined, for example, via Bootstrap Aggregation. A multivariate logistic regression model may be fed as input (together with the biomarkers) to a decision tree algorithm, or vice versa, the node assignments of a decision tree model may be fed as input (together with the biomarkers) into multivariate logistic regression. Similarly, any of the models may be fed as one of the inputs (together with the biomarkers) to a Neural Network.

### Selecting and Monitoring a Treatment Regimen

Just as the potential causes of any particular nonspecific symptom may be a large and diverse set of conditions, the appropriate treatments for these potential causes may be equally large and diverse. However, once a diagnosis is obtained, the clinician can readily select a treatment regimen that is compatible with the diagnosis. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999. With regard to SIRS, sepsis, severe sepsis, and septic shock, recent guidelines provide additional information for the clinician. See, e.g., Dellinger et al., Crit. Care Med. 32: 858-73, 2004, which is hereby incorporated by reference in its entirety.

Primary treatments available to US clinicians are antibiotics and intensive care support such as ventilators and hemodialysis in cases of organ failure. Recent advances are leading to improvements in how severe sepsis patients are treated. Xigris (drotrecogin alfa [activated], also known as activated Protein C) has found use in cases of severe sepsis. The following treatments can be included in a sepsis therapy regimen:

Administration of intravenous antibiotic therapy;
maintenance of a central venous pressure of 8-12 mm Hg;
administration of crystalloids and/or colloids, preferably to maintain such a central venous pressure;
maintenance of a mean arterial pressure of ≧65 mm Hg;
administration of one or more vasopressors (e.g., norepinephrine, dopamine, and/or vasopressin) and/or vasodilators (e.g., prostacyclin, pentoxifylline, N-acetyl-cysteine);
administration of one or more corticosteroids (e.g., hydrocortisone);
administration of recombinant activated protein C; maintenance of a central venous oxygen saturation of ≧70%;
administration of transfused red blood cells to a hematocrit of at least 30%;
administration of one or more inotropics (e.g., dobutamine); and
administration of mechanical ventilation.

This list is not meant to be limiting. In addition, since the methods and compositions described herein provide prognostic information, the panels and markers of the present invention may be used to monitor a course of treatment. For example, improved or worsened prognostic state may indicate that a particular treatment is or is not efficacious.

### Assay Measurement Strategies

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. For separate or sequential assay of markers, suitable apparatuses include clinical laboratory analyzers such as the ELECSYS® (Roche), the AXSYM® (Abbott), the ACCESS® (Beckman), the ADVIA® CENTAUR® (Bayer) immunoassay systems, the NICHOLS ADVANTAGE® (Nichols Institute) immunoassay system, etc.

Preferably the markers are analyzed using an immunoassay, and most preferably sandwich immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

Preferred apparatuses perform simultaneous assays of a plurality of markers using a single test device. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

The use of immobilized antibodies specific for the markers is also contemplated by the present invention. The antibodies could be immobilized onto a variety of solid phase supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

Particularly preferred assay devices of the present invention will comprise, for one or more assays, a first antibody conjugated to a solid phase and a second antibody conjugated to a signal development element. Such assay devices are configured to perform a sandwich immunoassay for one or more analytes. These assay devices will preferably further comprise a sample application zone, and a flow path from the sample application zone to a second device region comprising the first antibody conjugated to a solid phase.

Flow of a sample along the flow path may be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Most preferably, sample applied to the sample application zone will contact both a first antibody conjugated to a solid phase and a second antibody conjugated to a signal development element along the flow path (sandwich assay format). Additional elements, such as filters to separate plasma or serum from blood, mixing chambers, etc., may be included as required by the artisan. Exemplary devices are described in Chapter 41, entitled “Near Patient Tests: TRIAGE® Cardiac System,” in The Immunoassay Handbook, 2^{nd} ed., David Wild, ed., Nature Publishing Group, 2001, which is hereby incorporated by reference in its entirety.

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

A panel consisting of the markers referenced above may be constructed to provide relevant information related to differential diagnosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W.B. Saunders and Company, p. 496).

The present invention also provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses. This can include instructions and/or parameters on a computer-readable medium for use (i) in correlating assay results to a positive and/or negative result for the diagnoses, prognoses, etc., described herein; and/or (ii) lot specific information, such as standard curves, expiration dates, etc. Other measurement strategies applicable to the methods described herein include chromatography (e.g., HPLC), mass spectrometry, receptor-based assays, and combinations of the foregoing.

A computer readable storage medium, for example, one or more solid state memory devices (ROM chips or other removable chip-based memories), removable computer disks, magnetic strips, RFID-type inductive labels, bar codes, etc., can be provided in the kit to deliver test-related information and data to a computer processor used with the immunoassay device(s). In addition to operating instructions, such storage media can also be used to provide other pertinent data to a computer processor to be used in controlling and calibrating the tests to be performed. For example, test software can include program instructions and/or parameters used to direct the performance of one or more assays and correlations, as described herein. This may include calibration curves utilized to perform the desired test, test software, expiration dates, as well as other program information and calibration and control information for the instrument. In the case where the CCL23 assays are used in a univariate manner, this may include one or more thresholds used to assign likelihood of a diagnosis or prognosis, based on an assay result. In the case of multivariate analyses, this may include parameters used to combine the results of multiple markers, and some threshold(s) to which the combined result is compared for assigning the likelihood of a diagnosis or prognosis.

### Selection of Antibodies

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

### EXAMPLES

The following examples serve to illustrate the present invention. These examples are in no way intended to limit the scope of the invention.

### Example 1

### CCL23 Assay Design

As noted above, CCL23 splice variant differs from CCL23 by replacement of R₄₆ by MLWRRKIGPQMTLSHAAG (SEQ ID NO:3), represented by underlining in the following sequence of the mature CCL23 protein (that is, with the signal sequence deleted):

```
(SEQ ID NO:1)
                10 20 30 40 50 60
RVTKDAETEF MMSKLPLENP VLLDMLWRRK IGPQMTLSHA AGFHATSADC CISYTPRSIP
                70 80 90 100 110 116
CSLLESYFET NSECSKPGVI FLTKKGRRFC ANPSDKQVQV CMRMLKLDTR IKTRKN.
```

In addition, certain N-terminally truncated forms are believed generated by cleavage of the mature CCL23 protein in rheumatoid arthritis. See, e.g., Berahovich et al., J. Immunol. 174: 7341-51, 2005.

The following assays were designed:

Assay 1: A “total” CCL23 assay using two antibodies that paired in a sandwich format, and were each directed to an epitope C-terminal to the splice variant insertion and common to both CCL23 and CCL23 splice variant. This assay recognizes the major N-terminal processed form of CCL23 (CCL23₂₂₋₉₉) generated by elastase cleavage of CCL23 and the corresponding truncated form of CCL23 splice variant if it exists.
Assay 2: A “full length” CCL23 assay using two antibodies that paired in a sandwich format, one of which is directed to the portion of CCL23 missing from the N-terminal processed CCL23₁₉₋₉₉, CCL23₂₂₋₉₉, CCL23₂₇₋₉₉, and CCL23₃₀₋₉₉ forms, and the second of which is directed to an epitope C-terminal to the splice variant insertion and common to both CCL23 and CCL23 splice variant. This assay does not recognize the major N-terminal processed form of CCL23 (CCL23₂₂₋₉₉) generated by elastase cleavage of CCL23, or the corresponding truncated forms of CCL23 splice variant if they exist. Accordingly, this assay is a “full length CCL23 immunoassay” as that term is defined above.
Assay 3: A CCL23 splice variant assay using two antibodies that paired in a sandwich format, one antibody specific for the splice variant insert, and one antibody directed to the C-terminal region common to both CCL23 and CCL23 splice variant. This assay recognizes the major N-terminal processed form of CCL23 (CCL23₂₂₋₉₉) generated by elastase cleavage of CCL23 and the corresponding truncated form of CCL23 splice variant if it exists. Accordingly, this assay is a “CCL23 splice variant immunoassay” as that term is defined above.
Assay 4: A CCL23-specific assay using two antibodies that paired in a sandwich format, one antibody specific for CCL23, and one antibody directed to the C-terminal region common to both CCL23 and CCL23 splice variant. This assay does not recognize CCL23 splice variant, or the major N-terminal processed form of CCL23 (CCL23₂₂₋₉₉) generated by elastase cleavage of CCL23, or the corresponding truncated forms of CCL23 splice variant if they exist, nor does it recognize CCL23 splice variant.
Assay 5: An assay specific for N-terminally truncated CCL23₂₂₋₉₉ using two antibodies that paired in a sandwich format, one antibody specific for CCL23, and one antibody directed to the truncated site. This assay does not recognize CCL23 splice variant.
Assay 6: An assay specific for N-terminally truncated CCL23₂₅₋₉₉ using two antibodies that paired in a sandwich format, one antibody specific for CCL23, and one antibody directed to the truncated site. This assay does not recognize CCL23 splice variant.

Antibodies common to the CCL23 and CCL23 splice variant proteins were selected from antibody phage libraries generated from spleens of mice immunized with the CCL23 protein obtained from Cell Sciences, Canton, Mass.

The splice variant specific antibody was selected from antibody phage libraries generated from the spleens of mice immunized with the peptide MLWRRKIGPQMTLSHAAGC (SEQ ID NO:7) obtained from Biopeptide, San Diego, Calif. and conjugated to KLH. This corresponds to the sequence of the splice variant insert with an additional C-terminal cysteine through which the conjugation occurs.

The CCL23-specific antibody was selected from antibody phage libraries generated from the spleens of mice immunized with the peptide RFHATSADC (SEQ ID NO: 8) obtained from Biopeptide, San Diego, Calif.) and conjugated to KLH.

For antibodies specific to truncated forms, antibodies were selected from antibody phage libraries generated from spleens of mice immunized with the CCL23 protein. Panning was performed using biotinylated truncated forms in the presence of excess uncleaved CCL23 to remove antibodies that would bind the full length protein. To obtain the truncated forms, CCL23 and the CCL23 splice variant were each digested by mixing 50 μL of 0.5 mg/mL CCL23 or the splice variant in 50 μL of 100 mM Tris pH7.5, 20 mM CaCl₂, and 2 μL of 1 μg/μL (0.004 Unit/μL) elastase and incubating for 1-2 hours at room temperature.

### Example 2

### Immunization of Mice with Antigens and Purification of RNA from Mice

Ten C57 mice (Charles River Laboratories, Wilmington, Mass.) are immunized by subcutaneous administration of 50 μg of immunogen mixed with 15 μg of Quil A adjuvant (Accurate Chemical and Scientific Corp, Westbury, N.Y.) in PBS, pH 7.4 on day 0. A subsequent immunization is performed on day 14 using the immunogen mixed with Quil A. On day 23, blood samples are obtained from the mice by retro-orbital plexus bleeds and serum IgG responses are determined by ELISA using biotinylated immunogen immobilized in separate wells via neutravidin (Reacti-Bind™ NeutrAvidin™-Coated Polystyrene Plates, Pierce, Rockford, Ill.). Five of the mice (group A) are given two consecutive boosts of 50 μg of immunogen administered via intraperitoneal injection on days 29 and 30. On day 32, these mice are sacrificed and spleens are harvested for RNA isolation as described below. A third immunization is performed on the remaining five mice (group B) on day 28 using the antigen mixed with Quil A. On day 37, blood samples are obtained and serum IgG responses determined as described above. Two consecutive boosts of 50 μg of immunogen are administered via intraperitoneal injection on days 42 and 43. On day 45, the mice are sacrificed.

Spleens are harvested, macerated, then added to a polypropylene tube containing 3 mL of lysis Buffer (RAI Buffer, Macherey-Nagel) and homogenized for 1 min using a roto-stator homogenizer (Omni International). The lysates are added to wells of a Nucleospin Robot-96 RNA plate (Macherery-Nagel) and total RNA purified using a Tecan Genesis Workstation (Tecan).

### Example 3

### Enrichment of Polyclonal Antibody Phage

Antibody phage are generally prepared as described in WO 03/068956, the contents of which are incorporated by reference herein in their entirety, including all tables, figures, and claims, from mice immunized with as described above using BS60 uracil template. Antibody phage samples are panned with avidin magnetic latex generally as described in Example 16 of U.S. Pat. No. 6,057,098. Nucleic acids from enriched antibody phage samples are subcloned into a plasmid expression vector and electroporated into E. coli to generate antibody libraries as generally described in WO 03/068956.

### Example 4

### Selection of Monoclonal Sandwich Pairs

Antibody libraries are streaked on separate agar plates. Colonies expressing monoclonal antibodies from each library are picked to inoculate 96-well block cultures and grown overnight in at 37° C. A semi-defined culture medium (Pack, P. et al., Bio/Technology 11: 1271-1277, 1993, supplemented with 0.3 g/L L-leucine, 0.3 g/L L-isoleucine, 12 g/L cascin enzymatic hydrolysate (ICN Biomedicals, Costa Mesa, Calif.), 12.5 g/L glycerol, and 10 μg/mL tetracycline) is used for growth of the block cultures and subsequent scale-up cultures. Aliquots of the overnight cultures are used to generate frozen cell banks, and to start serial replicate 96-well block cultures to express and purify the antibodies as generally described in WO 03/068956.

Purified antibodies are assayed for functional positives as follows: Wells in Neutravidin plates (Pierce) are incubated with biotinylated target polypeptide for 1 hour at room temperature and washed. The wells are incubated with the purified antibodies for 1 hour at room temperature, washed, and incubated with Goat Anti-Mouse Kappa-Alkaline Phosphatase (Southern Biotechnology Associates) for 1 hour at room temperature. After a final wash, Attophos substrate solution (Promega) is added to the wells to generate kinetic fluorescent signals that are measured in a plate reader. The signals are used to identify and characterize which antibodies had been functionally captured in the wells. Select antibodies are scaled-up in shake flask cultures and purified.

Aliquots of these purified antibodies are biotinylated for use as detect antibodies to screen for sandwich antibody partners as follows. The purified antibodies in 96-well blocks are incubated overnight at 4° C. in replicate wells in high-binding plates (Nunc) to serve as capture antibodies. The wells are subsequently incubated with blocking buffer for 1 hour at room temperature and washed. The replicate wells are incubated with either unlabeled target polypeptide or buffer alone for 1 hour at room temperature and washed. Biotinylated detection antibodies are incubated in the replicate wells for 1 hour at room temperature and washed. The wells are incubated with Neutravidin-Alkaline Phosphatase (Southern Biotechnology Associates) for 1 hour at room temperature, washed, and Attophos substrate solution added to the wells to generate kinetic fluorescent signals that are measured in a plate reader. The relative signals in the replicate wells incubated with target polypeptide and buffer alone are used to identify and characterize which capture antibodies had formed a positive sandwich assay with the biotinylated detect antibodies. Based on this screen, selected antibodies are scaled-up in shake flasks and purified.

### Example 5

### Subject Population and Sample Collection

Test subjects in disease categories were enrolled as part of a prospective sepsis study conducted by Biosite Incorporated at 10 clinical sites in the United States. Enrollment criteria were: age 18 or older and presenting with two or more SIRS criteria, and confirmed or suspected infection and/or lactate levels greater than 2.5 mmol/L. Exclusion criteria were: pregnancy, cardiac arrest, and patients under Do Not Resuscitate (DNR) orders. Samples were collected by trained personnel in standard blood collection tubes with EDTA as the anticoagulant. The plasma was separated from the cells by centrifugation, frozen, and stored at −20 C or colder until analysis. The plasma was frozen within 1 hour. Clinical histories are available for each of the patients to aid in the statistical analysis of the assay data. Patients were assigned a final diagnosis by a physician at the clinical site using the standard medical criteria in use at each clinical site. Patients were diagnosed as having systemic inflammatory response syndrome (SIRS), sepsis, severe sepsis, septic shock or multiple organ dysfunction syndrome (MODS).

Samples from apparently healthy blood donors were purchased from Golden West Golden West Biologicals, Inc., Temecula, Calif., and were collected according to a defined protocol. Samples were collected from normal healthy individuals with no current clinical suspicion or evidence of disease. Blood was collected by trained personnel in standard blood collection tubes with EDTA as the anticoagulant. The plasma was separated from the cells by centrifugation, frozen, and stored at −20 C or colder until analysis.

### Example 6

### Immunoassays

In general, for a sandwich immunoassay in microtiter plates, a monoclonal antibody directed against a selected analyte is biotinylated using N-hydroxysuccinimide biotin (NHS-biotin) at a ratio of about 5 NHS-biotin moieties per antibody. The antibody-biotin conjugate is then added to wells of a standard avidin 384 well microtiter plate, and antibody conjugate not bound to the plate is removed. This forms the “anti-marker” in the microtiter plate. Another monoclonal antibody directed against the same analyte is conjugated to alkaline phosphatase, for example using succinimidyl 4-[N-maleimidomethyl]-cyclohexane-1-carboxylate (SMCC) and N-succinimidyl 3-[2-pyridyldithio]propionate (SPDP) (Pierce, Rockford, Ill.).

Biotinylated antibodies are pipetted into microtiter plate wells previously coated with avidin and incubated for 60 min. The solution containing unbound antibody is removed, and the wells washed with a wash buffer, consisting of 20 mM borate (pH 7.42) containing 150 mM NaCl, 0.1% sodium azide, and 0.02% TWEEN®-20 surface active agent (ICI Americas). The plasma samples (e.g., 10 μL-20 μL) containing added HAMA inhibitors are pipeted into the microtiter plate wells, and incubated for 60 min. The sample is then removed and the wells washed with a wash buffer. The antibody-alkaline phosphatase conjugate is then added to the wells and incubated for an additional 60 min, after which time, the antibody conjugate is removed and the wells washed with a wash buffer. A substrate, (ATTOPHOS®, Promega, Madison, Wis.) is added to the wells, and the rate of formation of the fluorescent product is related to the concentration of the analyte in the sample tested.

For competitive immunoassays in microtiter plates, a murine monoclonal antibody directed against a selected analyte is added to the wells of a microtiter plate and immobilized by binding to goat anti-mouse antibody that is pre-absorbed to the surface of the microtiter plate wells (Pierce, Rockford, Ill.). Any unbound murine monoclonal antibody is removed after a 60 minute incubation. This forms the “anti-marker” in the microtiter plate. A purified polypeptide that is either the same as or related to the selected analyte, and that can be bound by the monoclonal antibody, is biotinylated as described above for the biotinylation of antibodies. This biotinylated polypeptide is mixed with the sample in the presence of HAMA inhibitors (human anti-mouse antibodies, or HAMA, are human immunoglobulins with specificity for mouse immunoglobulins; HAMA inhibitors may be used to reduce or eliminate false signals from these human immunoglobulins; see, e.g., Reinsberg, Clin. Biochem. 29:145-48, 1996), forming a mixture containing both exogenously added biotinylated polypeptide and any unlabeled analyte molecules endogenous to the sample. The amount of the monoclonal antibody and biotinylated marker added depends on various factors and is titrated empirically to obtain a satisfactory dose-response curve for the selected analyte.

This mixture is added to the microtiter plate and allowed to react with the murine monoclonal antibody for 120 minutes. After the 120 minute incubation, the unbound material is removed, and Neutralite-Alkaline Phosphatase (Southern Biotechnology; Birmingham, Ala.) is added to bind to any immobilized biotinylated polypeptide. Substrate (as described above) is added to the wells, and the rate of formation of the fluorescent product was related to the amount of biotinylated polypeptide bound, and therefore is inversely related to the endogenous amount of the analyte in the specimen.

### Example 7

### CCL23 Assays

For each assay, the specific biotinylated anti-CCL23 antibody (primary antibody) diluted into assay buffer (10 mM Tris, 150 mM NaCl, 1% BSA) to 2 μg/mL was added to a 384 Neutravidin coated plate (Pierce Product #NC19658) and allowed to incubate at room temperature for 1 hour. Wells were washed with wash buffer (20 mM Borate, 150 mM NaCl, 0.2% TWEEN®(D 20 surface active agent (ICI Americas)) and then samples and standards were added and allowed to incubate at room temperature for 1 hour. Wells again were washed and then specific fluorsceinated anti-CCL23 antibody (secondary antibody) diluted in assay buffer to 2 μg/mL was added and allowed to incubate at room temperature for 1 hour. Wells again were washed. Added anti-fluorscein antibody conjugated to alkaline phosphatase, diluted 1/2338 into assay buffer was added and allowed to incubate at room temperature for 1 hour. Finally substrate (Promega ATTOPHOS®) was added and plate was read immediately. All additions were 10 μL/well unless otherwise stated. The plates were washed 3 times between each addition, with 9 final washes.

Standards were prepared by spiking various specific forms of CCL23 antigens into a normal EDTA plasma patient pool at concentrations ranging from 0.39 to 12.5 ng/mL, including a neutralized 0, which is the EDTA plasma pool with excess concentration of each antibody used in the ELISA. Reading was performed using a Tecan Spectrafluor plus using in kinetic mode over 6 read cycles with excitation filter of 430 nm and an emission filter 570 nm. Slope of RFU/seconds was determined.

### Example 8

### Results

Samples were obtained 3 hrs after the time of enrollment in the study described above to identify subjects at an increased risk for progression to sepsis, severe sepsis, or septic shock, and in-hospital mortality. Patients were classified into groups as follows in Table 1. The categories EA and AS together are referred to herein by the term “advanced sepsis.”

TABLE 1

| Category | Description |
|---|---|
| Normal | Banked samples from normal healthy donors. |
| SL | Patients enrolled in the study with low risk infection |
| | (or no infection) and with 2 or more SIRS criteria upon |
| | presentation to the ED. |
| ES | Patients with a high risk infection and 2 or more SIRS |
| | criteria, but not meeting the criteria for Severe Sepsis, |
| | or Septic Shock within 72 hrs of presentation to the ED. |
| EA | Patients that did not meet the criteria for Severe Sepsis, |
| | or Septic Shock upon presentation to the ED, but advanced |
| | to meet these criteria within 72 hrs. |
| AS | Patients meeting the criteria for Severe Sepsis, or Septic |
| | Shock upon presentation to the ED. |
| Alive | Patients in categories ES, EA, or AS who did not die in |
| | the hospital. |
| Dead | Patients in categories ES, EA, or AS who died in the hospital. |

The data was analyzed using standard Receiver Operator Characteristic (ROC) analysis for the case discrimination criteria shown in Table 2. “N” refers to the number of individuals in a respective category.

TABLE 2

| Criteria (written as | | | |
|---|---|---|---|
| Category(0) vs. | N | N | |
| Category(1)) | Category(0) | Category(1) | Description of comparison |
| Alive vs. Dead | 108 | 47 | Risk of death in patients with Sepsis. |
| EA vs. AS | 36 | 83 | Risk of progression to Severe Sepsis, |
| | | | or Septic Shock in patients with |
| | | | Sepsis. |
| ES + EA vs. AS | 72 | 83 | Risk of progression to Severe Sepsis, |
| | | | or Septic Shock in patients with |
| | | | Sepsis. |
| ES vs. EA | 36 | 36 | Risk of progression to Severe Sepsis, |
| | | | or Septic Shock in patients with |
| | | | Sepsis. |
| SL + ES vs. EA + AS | 140 | 119 | Risk of Progression to Sepsis |
| SL vs. AS | 104 | 83 | Diagnosis of Severe Sepsis, or Septic |
| | | | Shock |
| SL vs. ES + EA + AS | 104 | 155 | Risk of Progression to Sepsis |
| SL vs. ES + EA | 104 | 72 | Risk of Progression to Sepsis |
| SL vs. ES | 104 | 36 | Risk of Progression to Sepsis |
| Normal vs. ES + EA + | 23 | 155 | Diagnosis of Sepsis |
| AS | | | |

ROC AUC (area under the curve) were calculated for each of the assays (numbered as in Example 1 above) in each of the discrimination criteria. The statistical significance of each ROC AUC was calculated assuming a null hypothesis that the true ROC AUC is 0.5 (which is the ROC AUC of a random test, i.e., an assay that has no ability to discriminate the criteria). P values for statistical significance were calculated using a standard Z-test. A result was significant if p≦0.05; NS refers to results that were not significant. The results are shown in Tables 3 and 4.

TABLE 3

| | ROC Area for Assay | | | | | |
|---|---|---|---|---|---|---|
| | Assay | Assay | Assay | Assay | Assay | Assay |
| Criteria | 2 | 4 | 5 | 6 | 3 | 1 |
| Alive vs. Dead | 0.57 | 0.55 | 0.47 | 0.44 | 0.60 | 0.60 |
| EA vs. AS | 0.59 | 0.58 | 0.52 | 0.51 | 0.65 | 0.63 |
| ES + EA vs. AS | 0.63 | 0.63 | 0.48 | 0.47 | 0.68 | 0.68 |
| ES vs. EA | 0.55 | 0.59 | 0.42 | 0.42 | 0.54 | 0.57 |
| SL + ES vs. EA + AS | 0.75 | 0.77 | 0.51 | 0.47 | 0.79 | 0.80 |
| SL vs. AS | 0.81 | 0.84 | 0.54 | 0.49 | 0.87 | 0.87 |
| SL vs. ES | 0.72 | 0.72 | 0.60 | 0.56 | 0.71 | 0.74 |
| SL vs. ES + EA | 0.73 | 0.74 | 0.56 | 0.52 | 0.72 | 0.76 |
| SL vs. ES + EA + AS | 0.77 | 0.79 | 0.55 | 0.50 | 0.80 | 0.82 |
| Normal vs. ES + EA + AS | 0.99 | 0.97 | 0.66 | 0.66 | 0.96 | 0.99 |

TABLE 4

| | P value for Assay | | | | | |
|---|---|---|---|---|---|---|
| | Assay | Assay | Assay | Assay | Assay | Assay |
| Criteria | 2 | 4 | 5 | 6 | 3 | 1 |
| Alive vs. Dead | NS | NS | NS | NS | <0.05 | <0.05 |
| EA vs. AS | NS | NS | NS | NS | <0.05 | <0.05 |
| ES + EA vs. AS | <0.01 | <0.01 | NS | NS | <0.001 | <0.001 |
| ES vs. EA | NS | NS | NS | NS | NS | NS |
| SL + ES vs. EA + AS | <0.001 | <0.001 | NS | NS | <0.001 | <0.001 |
| SL vs. AS | <0.001 | <0.001 | NS | NS | <0.001 | <0.001 |
| SL vs. ES | <0.001 | <0.001 | NS | NS | <0.001 | <0.001 |
| SL vs. ES + EA | <0.001 | <0.001 | NS | NS | <0.001 | <0.001 |
| SL vs. ES + EA + AS | <0.001 | <0.001 | NS | NS | <0.001 | <0.001 |
| Normal vs. ES + EA + AS | <0.001 | <0.001 | <0.01 | <0.01 | <0.001 | <0.001 |

The following conclusions were drawn from this data.

(1) The truncated form-specific assays that exclude splice variant have no discriminatory power in this patient population.

(2) The “total” CCL23 assay that recognizes CCL23, CCL23 splice variant, and possible truncated forms perform comparable to the “full length” CCL23 assay that recognizes CCL23 and CCL23 splice variant, but not the truncated forms in a paired comparison.

(3) The CCL23 splice variant assay performs comparably to the total CCL23 assay that recognizes CCL23 and CCL23 splice variant, but not the truncated forms in a paired comparison. The CCL23-specific assay that does not recognize the CCL23 splice variant also performs comparably to the total CCL23 assay that recognizes CCL23 and CCL23 splice variant.

Given the relative performance of assays 1, 2, and 3, odds ratios were calculated to demonstrate the relative performance of these assays to distinguish subjects at risk to progress to the more severe sepsis categories. The following data presents odds rations for the relative risk of falling into the following two groups: Outcome 0: patients enrolled in the study with low risk infection (or no infection) and with 2 or more SIRS criteria upon presentation to the ED; outcome 1: patients with a high risk infection and 2 or more SIRS criteria, but not meeting the criteria for severe sepsis, or septic shock within 72 hrs of presentation to the ED, or patients that did not meet the criteria for severe sepsis, or septic shock upon presentation to the ED, but advanced to meet these criteria within 72 hrs, or patients meeting the criteria for severe sepsis, or septic shock upon presentation to the ED. In Table 5, N is the number of patients whose samples were analyzed within each group, odds ratios are calculated relative to the first quartile, 95% LCI is the lower 95% confidence interval of each odds ratio, and 95% UCI is the upper 95% confidence interval of each odds ratio.

Quartiles

| | 1st | 2nd | 3rd | 4th |
|---|---|---|---|---|
| Assay 1 | | | | |
| N (Outcome 0) | 49 | 35 | 13 | 7 |
| N (Outcome 1) | 16 | 30 | 51 | 58 |
| Total N | 65 | 65 | 64 | 65 |
| Odds Ratio | 1.00 | 2.63 | 12.01 | 25.38 |
| 95% LCI | n/a | 1.25 | 5.24 | 9.66 |
| 95% UCI | n/a | 5.53 | 27.56 | 66.68 |
| CCL23 | n/a | 3.88 | 6.14 | 10.25 |
| concentration at | | | | |
| lower interval | | | | |
| boundary (ng/mL) | | | | |
| Assay 2 | | | | |
| N (Outcome 0) | 47 | 34 | 12 | 11 |
| N (Outcome 1) | 18 | 31 | 52 | 54 |
| Total N | 65 | 65 | 64 | 65 |
| Odds Ratio | 1.00 | 2.38 | 11.31 | 12.82 |
| 95% LCI | n/a | 1.15 | 4.93 | 5.50 |
| 95% UCI | n/a | 4.94 | 25.95 | 29.87 |
| CCL23 | n/a | 1.46 | 3.07 | 5.53 |
| concentration at | | | | |
| lower interval | | | | |
| boundary (ng/mL) | | | | |
| Assay 3 | | | | |
| N (Outcome 0) | 47 | 36 | 14 | 7 |
| N (Outcome 1) | 18 | 29 | 50 | 58 |
| Total N | 65 | 65 | 64 | 65 |
| Odds Ratio | 1.00 | 2.10 | 9.33 | 21.63 |
| 95% LCI | n/a | 1.01 | 4.17 | 8.33 |
| 95% UCI | n/a | 4.37 | 20.84 | 56.17 |
| CCL23 | n/a | 0.29 | 0.48 | 0.80 |
| concentration at | | | | |
| lower interval | | | | |
| boundary (ng/mL) | | | | |

While all three of these assays perform acceptably, Assay 1, which is the total CCL23 assay that recognizes CCL23, CCL23 splice variant, and possible truncated forms, may be superior, although there are not enough patient samples to demonstrate the statistical significance of this observation.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A method of diagnosing SIRS, sepsis, severe sepsis, septic shock, or MODS in a subject, or assigning a prognostic risk for one or more clinical outcomes for a subject suffering from SIRS, sepsis, severe sepsis, septic shock, or MODS, the method comprising:
performing an immunoassay that detects CCL23 splice variant (SEQ ID NO: 1), to provide an immunoassay result; and
relating the immunoassay result to one or more diagnoses or prognoses selected from the group consisting of the presence or absence of SIRS, the presence or absence of sepsis, the presence or absence of severe sepsis, the presence or absence of septic shock, and the prognostic risk of one or more clinical outcomes for the subject suffering from or believed to suffer from SIRS, sepsis, severe sepsis, septic shock, or MODS.

2. A method according to claim 1, wherein said immunoassay that detects CCL23 splice variant does not appreciably detect CCL23 (SEQ ID NO: 2).

3. A method according to claim 1, wherein said immunoassay that detects CCL23 splice variant also detects CCL23.

4. A method according to claim 3, wherein said assay provides a signal that is within a factor of 3 for CCL23 splice variant at a concentration between 3 ng/mL and 10 ng/mL as compared to equimolar amounts of CCL23.

5. A method according to claim 3, wherein said assay provides a signal that is within a factor of 2 for CCL23 splice variant at a concentration between 3 ng/mL and 10 ng/mL as compared to equimolar amounts of CCL23.

6. A method according to claim 3, wherein said assay provides a signal that is within a factor of 0.5 for CCL23 splice variant at a concentration between 3 ng/mL and 10 ng/mL as compared to equimolar amounts of CCL23.

7. A method according to claim 3, wherein said assay also detects one or more fragments of CCL23 that lack N-terminal residues removed from CCL23 by elastase.

8. A method according to claim 3, wherein said assay also detects one or more fragments of CCL23 residues deleted for up to 30 residues from the N-terminus of SEQ ID NO: 2.

9. A method according to claim 1, selected from the group consisting of:
(1) a sandwich format immunoassay using two antibodies, each of which binds to an epitope common to both CCL23 and CCL23 splice variant;
(2) a sandwich format immunoassay using two antibodies, one of which binds to the portion of CCL23 missing from one or more of N-terminal processed forms of CCL23 selected from the group consisting of CCL23₁₉₋₉₉, CCL23₂₂₋₉₉, CCL23₂₇₋₉₉, and CCL23₃₀₋₉₉, and the second of which binds to an epitope common to both CCL23 and CCL23 splice variant;
(3) a sandwich format immunoassay using two antibodies, one of which binds to CCL23 splice variant but not CCL23, and the second of which binds to an epitope common to both CCL23 and CCL23 splice variant; and
(4) a sandwich format immunoassay using two antibodies, each of which binds to CCL23 splice variant but not CCL23.

10. A method according to claim 9, wherein at least one antibody binds to an epitope common to both CCL23 and CCL23 splice variant that is C-terminal to the splice variant insertion MLWRRKIGPQMTLSHAAG (SEQ ID NO:3).

11. A method according to claim 9, wherein each antibody in the sandwich immunoassay binds to an epitope common to both CCL23 and CCL23 splice variant, and each antibody in the sandwich immunoassay binds one or more of N-terminal processed forms of CCL23 selected from the group consisting of CCL23₁₉₋₉₉, CCL23₂₂₋₉₉, CCL23₂₇₋₉₉, and CCL23₃₀₋₉₉.

12. A method according to claim 1, wherein said assay method comprises performing one or more additional immunoassays that detect markers selected from the group consisting of NT-proBNP, proBNP, BNP₇₉₋₁₀₈, BNP, BNP₃₋₁₀₈, CCL23, CRP, D-dimer, IL-1ra, NGAL, peptidoglycan recognition protein, procalcitonin, procalcitonin₃₋₁₁₆, active protein C, latent protein C, total protein C, and sTNFR1a to provide one or more additional immunoassay results, and wherein said relating step comprises relating the immunoassay result and the one or more additional immunoassay results to one or more diagnoses or prognoses selected from the group consisting of the presence or absence of SIRS, the presence or absence of sepsis, the presence or absence of severe sepsis, the presence or absence of septic shock, and the prognostic risk of one or more clinical outcomes for the subject suffering from or believed to suffer from SIRS, sepsis, severe sepsis, septic shock, or MODS.

13. A method according to claim 1, wherein the relating step comprises relating the immunoassay result to a prognostic risk of mortality.

14. A method according to claim 1, wherein the relating step comprises relating the immunoassay result to a prognostic risk of mortality.

15. A method according to claim 1, wherein said relating step comprises comparing the immunoassay result to a predetermined threshold level selected to provide a sensitivity or specificity of at least 0.7 for the diagnosis of sepsis, compared to SIRS not progressed to sepsis.

16. A method according to claim 1, wherein said relating step comprises comparing the immunoassay result to a predetermined threshold level selected to provide a sensitivity or specificity of at least 0.7 for the diagnosis of severe sepsis, compared to SIRS not progressed to severe sepsis.

17. A method according to claim 1, wherein said relating step comprises comparing the immunoassay result to a predetermined threshold level selected to provide a sensitivity or specificity of at least 0.7 for the diagnosis of septic shock, compared to SIRS not progressed to septic shock.

18. A method according to claim 1, wherein said relating step comprises comparing the immunoassay result to a predetermined threshold level selected to provide an odds ratio of at least 2 for the prognostic risk of mortality.

19. A method according to claim 1, wherein the sample is selected from the group consisting of blood, serum, and plasma.

20. A method of formulating a CCL23 immunoassay for use in assessing a subject with SIRS, comprising:
providing an antibody pair for use in a sandwich format immunoassay, wherein said antibody pair is selected from the group consisting of:
(1) a first and a second antibody, each of which binds to an epitope common to both CCL23 and CCL23 splice variant;
(2) a first antibody that binds to the portion of CCL23 missing from one or more of N-terminal processed forms of CCL23 selected from the group consisting of CCL23₁₉₋₉₉, CCL23₂₂₋₉₉, CCL23₂₇₋₉₉, and CCL23₃₀₋₉₉, and a second antibody that binds to an epitope common to both CCL23 and CCL23 splice variant;
(3) a first antibody that binds to CCL23 splice variant but not CCL23, and a second antibody that binds to an epitope common to both CCL23 and CCL23 splice variant; and
(4) a first and a second antibody, each of which binds to CCL23 splice variant but not CCL23; and
measuring a sample obtained from a subject having SIRS using said antibody pair in a sandwich format immunoassay.

21. A method according to claim 20, wherein at least one of said first and second antibodies binds to an epitope common to both CCL23 and CCL23 splice variant that is C-terminal to the splice variant insertion MLWRRKIGPQMTLSHAAG (SEQ ID NO:3).

22. A method according to claim 20, wherein each of said first and second antibodies binds to an epitope common to both CCL23 and CCL23 splice variant, and each of said first and second antibodies binds one or more of N-terminal processed forms of CCL23 selected from the group consisting of CCL23₁₉₋₉₉, CCL23₂₂₋₉₉, CCL23₂₇₋₉₉, and CCL23₃₀₋₉₉.

23. A method according to claim 20, wherein one of said first or second antibodies is conjugated to a solid phase, and the other of said first or second antibodies is conjugated to a detectable label.

24. A kit comprising:
reagents for performing a sandwich format immunoassay, wherein said reagents comprise an antibody pair selected from the group consisting of:
(1) a first and a second antibody, each of which binds to an epitope common to both CCL23 and CCL23 splice variant;
(2) a first antibody that binds to the portion of CCL23 missing from one or more of N-terminal processed forms of CCL23 selected from the group consisting of CCL231999, CCL23₂₂₋₉₉, CCL23₂₇₋₉₉, and CCL23₃₀₋₉₉, and a second antibody that binds to an epitope common to both CCL23 and CCL23 splice variant;
(3) a first antibody that binds to CCL23 splice variant but not CCL23, and a second antibody that binds to an epitope common to both CCL23 and CCL23 splice variant; and
(4) a first and a second antibody, each of which binds to CCL23 splice variant but not CCL23;
wherein one of said first or second antibodies is conjugated to a solid phase, and the other of said first or second antibodies is conjugated to a detectable label; and
a computer readable medium comprising instructions, parameters, or both to be read by a computer processor and used by said processor in relating results of an immunoassay performed using said reagents to a concentration of CCL23 or a related marker in a test sample.

25. A kit according to claim 24, wherein said instructions, parameters, or both comprise one or more of: encoded standard data for relating a detectable signal generated from said detectable label to a concentration of CCL23 or a related marker; an encoded expiration date for said kit; and encoded data that causes the processor to calculate said concentration of CCL23 or a related marker and to compare said concentration of CCL23 or a related marker, either alone or in combination with one or more other marker results, to a threshold indicative of a likelihood of one or more diagnoses or prognoses selected from the group consisting of the presence or absence of SIRS, the presence or absence of sepsis, the presence or absence of severe sepsis, the presence or absence of septic shock, and the prognostic risk of one or more clinical outcomes for the subject suffering from or believed to suffer from SIRS, sepsis, severe sepsis, septic shock, or MODS.

26. A method of assigning a prognostic risk of sepsis progression to a subject suffering from SIRS, the method comprising:
performing an assay method on one or more samples obtained from said subject, wherein said assay method comprises performing a plurality of immunoassays that detect CCL23 splice variant (SEQ ID NO: 1), NGAL, and C-reactive protein to provide a plurality of immunoassay results; and
relating the immunoassay results obtained from said assay method to the prognostic risk of sepsis progression for the subject.

27. A method according to claim 26, wherein said immunoassay that detects CCL23 splice variant is selected from the group consisting of:
(1) a sandwich format immunoassay using two antibodies, each of which binds to an epitope common to both CCL23 and CCL23 splice variant;
(2) a sandwich format immunoassay using two antibodies, one of which binds to the portion of CCL23 missing from one or more of N-terminal processed forms of CCL23 selected from the group consisting of CCL23₁₉₋₉₉, CCL23₂₂₋₉₉, CCL23₂₇₋₉₉, and CCL23₃₀₋₉₉, and the second of which binds to an epitope common to both CCL23 and CCL23 splice variant;
(3) a sandwich format immunoassay using two antibodies, one of which binds to CCL23 splice variant but not CCL23, and the second of which binds to an epitope common to both CCL23 and CCL23 splice variant; and
(4) a sandwich format immunoassay using two antibodies, each of which binds to CCL23 splice variant but not CCL23.

28. A method according to claim 27, wherein said prognostic risk is a risk of sepsis progression within 72 hours of obtaining one or more of said samples.

29. A method according to claim 27, wherein said prognostic risk of sepsis progression is a risk of said patient having one or more conditions selected from the group consisting of a high risk infection, severe sepsis, and septic shock within 72 hours of obtaining one or more of said samples.

30. A method according to claim 27, wherein said immunoassay results are used to calculate a single value that is a function of each of the immunoassay results obtained from said assay method, and said single value is compared to a threshold value;
wherein when said single value is greater than said threshold value, said subject is assigned an increased risk of sepsis progression relative to a risk assigned when said single value is less than said threshold value.
